(19) United States
(12) Patent Application Publication
(10) Pub. No.: US 2023/0070921 A1
(43) Pub. Date: Mar. 9, 2023

(54) Reconstruction Algorithms for DNA-Storage Systems

(71) Applicant: TECHNION RESEARCH AND DEVELOPMENT FOUNDATION LTD., HAIFA (IL)

(72) Inventors: OMER SABARY, KIRYAT ATTA (IL); GUY SHAPIRA, KIRYAT MOTSKIN (IL); EITAN YAAKOBI, TEL AVIV (IL); ALEXANDER YUCOVICH, NESHER (IL)

(73) Assignee: TECHNION RESEARCH AND DEVELOPMENT FOUNDATION LTD., HAIFA (IL)

(21) Appl. No.: 17/447,066

(22) Filed: Sep. 8, 2021

Related U.S. Application Data
(60) Provisional application No. 63/075,753, filed on Sep. 8, 2020.

(51) Int. Cl. G16B 50/30; G06N 3/12

(52) U.S. Cl. CPC G16B 50/30; G06N 3/123

(57) ABSTRACT

There may be provided method for estimating an information unit represented by a cluster of traces that are noisy copies of a synthesized strand, the method may include estimating the information unit by applying processing operations on r-tuples related to the traces, wherein r is smaller than a number (t) of traces of the cluster; wherein processing operations applied on at least some of the r-tuples comprise calculating a length of a shortest common supersequences (SCS) of the r-tuples.

### BACKGROUND

Recent studies presented a significant progress in DNA synthesis and sequencing technologies. This progress also introduced the development of data storage technology based upon DNA molecules. A DNA storage system consists of three important components. The first is the DNA synthesis which produces the oligonucleotides, also called strands, that encode the data. In order to produce strands with accept able error rates, in a high throughput manner, the length of the strands is typically limited to no more than 250 nucleotides. The second part is a storage container with compartments which stores the DNA strands, however without order. Finally, sequencing is performed to read back a representation of the strands, which are called reads.

Current synthesis technologies are not able to generate a single copy for each DNA strand, but only multiple copies where the number of copies is in the order of thousands to millions. Moreover, sequencing of DNA strands is usually preceded by PCR amplification which replicates the strands. Hence, every strand has multiple copies and several of them are read during sequencing.

The encoding and decoding stages are two processes, external to the storage system, that convert the user's binary data into strands of DNA such that, even in the presence of errors, it will be possible to revert back and recover the original binary data. These two stages consist of three steps, which we refer by 1. clustering, 2. reconstruction, and finally 3. error correction. After the strands are read back by sequencing, the first task is to partition them into clusters such that all strands in the same cluster originated from the same synthesized strand. After the clustering step, the goal is to reconstruct each strand based upon all its noisy copies, and this stage is the main problem studied in this paper. Lastly, errors which were not corrected by the reconstruction step, mis-clustering errors, lost strands, and any other error mechanisms should be corrected by the use of an error-correcting code.

Any reconstruction algorithm for the second stage is performed on each cluster to recover the original strand from the noisy copies in the cluster. Having several copies for each strand is beneficial since it allows to correct errors that may occur during this process. In fact, this setup falls under the general framework of the string reconstruction problem which refers to recovering a string based upon several noisy copies of it. Examples for this problem are the sequence reconstruction problem which was first studied by Levenshtein and the trace reconstruction problem. In general, these models assume that the information is transmitted over multiple channels, and the decoder, which observes all channel estimations, uses this inherited redundancy in order to correct the errors.

Generally speaking, the main problem studied under the paradigm of the sequence reconstruction and trace reconstruction problems is to find the minimum number of channels that guarantee successful decoding either in the worst case or with high probability. However, in DNA-based storage systems we do not necessary have control on the number of strands in each cluster. Hence, the goal of this work is to propose efficient algorithms for the reconstruction problem as it is reflected in DNA-based storage systems where the cluster size is a given parameter. Then, the goal is to output a strand that is close to the original one so that the number of errors the error-correcting code should correct will be minimized. We will present algorithms that work with a flexible number of copies and various probabilities for deletion, insertion, and substitution errors.

### 0.1 DNA Storage and DNA Errors

One of the early experiments of data storage in DNA was conducted by Clellan et al. in 1999. In their study they coded and recovered a message consisting of 23 characters. Three sequences of nine bits each, have been successfully stored by Leier et al. in 2000. Gibson et al. presented in 2010 a more significant progress, in terms of the amount of data stored successfully. They demonstrated in-vivo storage of 1,280 characters in a bacterial genome. The first large scale demonstrations of the potential of in vitro DNA storage were reported by Church et al. who recovered 643 KB of data and by Goldman et al. who accomplished the same task for a 739 KB message. However, both of these pioneering groups did not recover the entire message successfully and no error correcting codes were used. Shortly later, Grass et al. have managed to successfully store and recover a 81 KB message, in an encapsulated media, and Bornholt et al. demonstrated storing a 42 KB message. A significant improvement in volume was reported in by Blawat et al. who successfully stored 22 MB of data. Erlich and Zielinski improved the storage density and stored 2.11 MB of data. The largest volume of stored data was reported by Organick et al. who stored roughly 200 MB of data, an order of magnitude more data than previously reported. Yazdi et al. developed a method that offers both random access and rewritable storage and in a portable DNA-based storage system. Recently, Anavy et al. enhanced the capacity of the DNA storage channel by using composite DNA letter. A similar approach, on a smaller scale. Lopez et al. stored and decoded a 1.67 MB of data. In their work they focused on increasing the throughput of nanopore sequencing by assembling and sequencing together fragments of 24 short DNA strands. Recent studies also presented an end-to-end demonstration of DNA storage, the use of LDPC codes for DNA-based storage, a computer systems prospective on molecular processing and storage, and lastly, the work of Tabatabaei et al. which uses existing DNA strands as punch cards to store information.

The processes of synthesizing, storing, sequencing, and handling strands are all error prone. Each step in these processes can independently introduce a significant number of errors. Additionally, the DNA storage channel has several attributes which distinguish it from other storage media such as tapes, hard disk drives, and flash memories. We summarize some of these differences and the special error behavior in DNA.
1. Both the synthesis and sequencing processes can introduce deletion, insertion, and substitution errors on each of the read and synthesized strands.
2. Current synthesis methods cannot generate one copy for each design strand. They all generate thousands to millions of noisy copies, while different copies may have a different error distribution. Moreover, some strands may have a significant larger number of copies, while some other strands may not have copies all.
3. The use of DNA for storage or other applications typically involves PCR amplification of the strands in the DNA pool. PCR is known to have a preference for some strands over others, which may further distort the distribution of the number of copies of individual strands and their error profiles.
4. Longer DNA strands can be sequenced using designated sequencing technologies, e.g. PacBio and Oxford Nanopore. However, the error rates of these technologies can be significantly higher and can grow up to 30%, with deletions and substitutions as the most dominant errors.

### 0.2 BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 illustrates an example of Levenshtein errors;

FIG. 2 illustrate an example of k0error success rates;

FIG. 3 illustrate an example of k-mer distances;

FIG. 4 illustrate an example of performances;

FIG. 5 illustrate an example of patterns of y1;

FIG. 6 illustrate an example of exit error rates;

FIG. 7 illustrate an example of success rate by error probabilities;

FIG. 8 illustrate an example of k1ₛuccvalues;

FIG. 9 illustrate an example of edit error rates;

FIG. 10 is an example of a method;

FIG. 11 is an example of a method;

FIG. 12 is an example of a table;

FIGS. 13 and 14 are examples of estimated conditional probabilities;

FIG. 15 is an example of a second table and a graph related to a symbol located at a certain location;

FIG. 16 is an example of a method for finding the heaviest path;

FIG. 17 is an example of a method for finding a conditional letter probability; and

FIG. 18 illustrates an example of a method for estimating an information unit.

### 0.3 DETAILED DESCRIPTION OF THE DRAWINGS

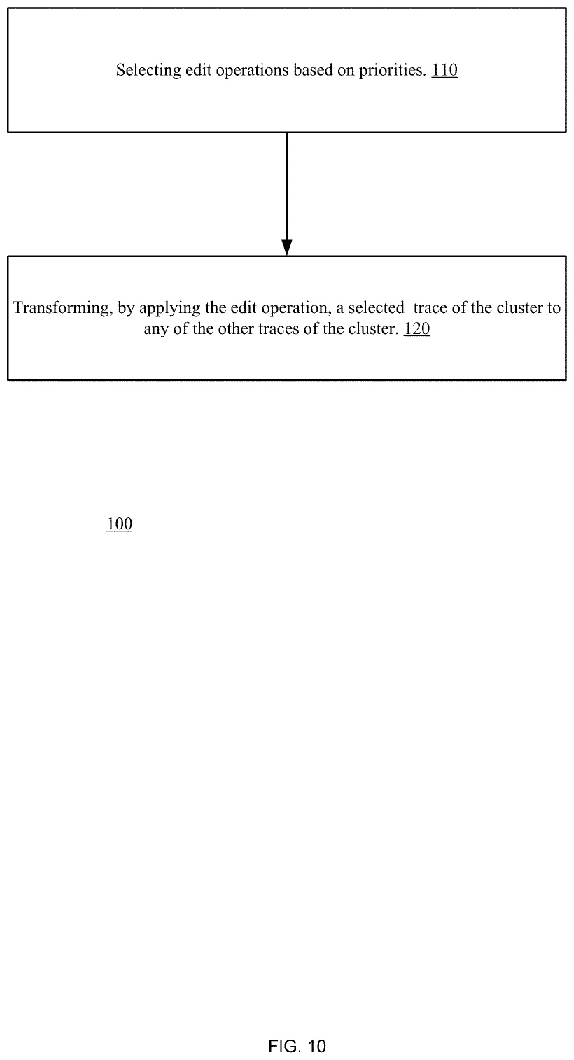

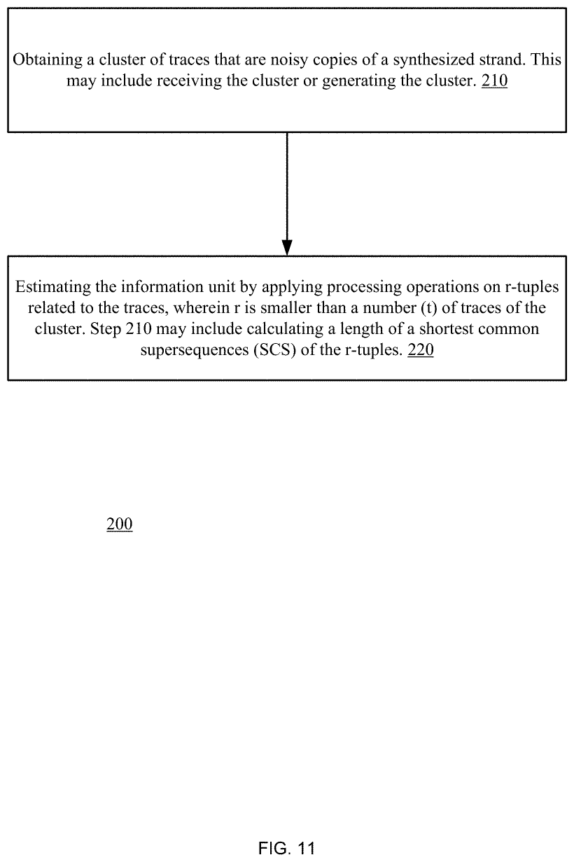

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings. It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements. Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention. Any reference in the specification to a method should be applied mutatis mutandis to a device or system capable of executing the method and/or to a non-transitory computer readable medium that stores instructions for executing the method. Any reference in the specification to a system or device should be applied mutatis mutandis to a method that may be executed by the system, and/or may be applied mutatis mutandis to non-transitory computer readable medium that stores instructions executable by the system. Any reference in the specification to a non-transitory computer readable medium should be applied mutatis mutandis to a device or system capable of executing instructions stored in the non-transitory computer readable medium and/or may be applied mutatis mutandis to a method for executing the instructions. Any combination of any module or unit listed in any of the figures, any part of the specification and/or any claims may be provided. The specification and/or drawings may refer to a processor. The processor may be a processing circuitry. The processing circuitry may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits. Any combination of any steps of any method illustrated in the specification and/or drawings may be provided. Any combination of any subject matter of any of claims may be provided. Any combinations of systems, units, components, processors, sensors, illustrated in the specification and/or drawings may be provided. There may be provided systems, methods and a non-transitory computer readable media for reconstruction of data stored in a DNA storage system. The following text refers to methods for simplicity of explanation. There may be provided methods that reconstructs the data using shortest common supersequences (SCS) and Longest Common Subsequences (LCS). In contrary to most of the previous algorithms that are shaped in the structure of alignment then majority reconstruction—the methods also involves finding the SCS and LCS, identifying similar patterns in the sequences, and using the maximum-likelihood decoder. The methods may use the edit distances between the traces as the basis of the estimation. Instead of “building” the estimation from sketch, the methods may use the edit distances, and error vectors between couple of traces, in order to align them, and then using one or more them as the basis of our output. The methods may be determined based on practical values of DNA storage system—by taking into account an error characterization of the DNA storage channel. The methods places less limitations on the input. While most previous algorithms support limited number of errors, dependency between the errors, limited lengths of the sequence or limited number of distinct traces etc., the methods is flexible, and consider any error distribution, any strands length. Minimization of the error rates and maximization of the success rate. The methods was designed for practical purposes. Hence, it aims on reducing the error rates of the reconstructed sequences and maximizing the success rate, which is the probability of exact reconstruction. The inventors evaluated the distribution of number of errors of the reconstructed sequences. Using these values may be beneficial as they define the parameters of the error correcting codes to guarantee exact reconstruction in a given library. FIG. 10 is an example of method 100 for estimating an information unit represented by a cluster of traces that are noisy copies of a synthesized strand. Method 100 may start by step 110 of selecting edit operations based on priorities. Step 110 may be followed by step 120 of transforming, by applying the edit operation, a selected trace of the cluster to any of the other traces of the cluster. Step 110 may include or may be preceded by performing a majority vote of the edit operations in order correct errors in the selected trace. The method (for example step 120) may include computing an LCS of two traces, performing a majority vote on indices of each symbol of the LCS in each of the traces in the cluster, and setting an outcome of the majority votes as anchors in the estimated string. The method (for example step 120) may include computing set of patterns of pairs of traces in the cluster using their LCSs and their error vectors. The estimated information unit may be one of the revised and corrected traces drives mentioned above, and is selected based upon its length and its frequency The methods may support high success rate with smaller cluster size. Compared to the previously published algorithms, the methods increases the probability of exact reconstruction while using less traces from each clusters. The method may assume that the clustering step has been done successfully. This could be achieved by the use of indices in the strands and other advanced coding techniques. Thus, the input to the algorithms is a cluster of noisy read strands, and the goal is to efficiently output the original strand or a close estimation to it with high probability. FIG. 11 illustrates method 200 for estimating an information unit represented by a cluster of traces that are noisy copies of a synthesized strand. Method 200 may start by step 210 of obtaining a cluster of traces that are noisy copies of a synthesized strand. This may include receiving the cluster or generating the cluster. Step 210 may be followed by step 220 of estimating the information unit by applying processing operations on r-tuples related to the traces, wherein r is smaller than a number (t) of traces of the cluster. Step 210 may include calculating a length of a shortest common supersequences (SCS) of the r-tuples.

Step 220 may include applying a processing operation on at least some of the r-tuples—wherein the applying may include searching for a maximum likelihood SCS.When not finding, the maximum likelihood SCS, then returning a SCS that minimizes a sum of Levenshtein distances of all the traces of the cluster.

Step 220 may include repeating the processing operations for different values of r. The value of r may or may not exceed ten.

There may be only a few different values of r. A few—may not exceed 5, 6, 7, 8, 9, 10 and the like.

The processing operations applied on the at least some of the r-tuples may include calculating longest common subsequences (LCSs).

Step 220 of estimating the information unit may be based on a size of the cluster. Step 220 of estimating may include estimating an error probability of the cluster using an average length of the traces.

Step 220 of estimating may include applying the processing operations only on a group of longest traces of the cluster. The longest traces may be about one fifth of the traces of the cluster. Step 220 may include calculating a distance between the traces of the cluster. The calculating of the distance between the traces of the cluster may be followed by the processing of a r-tuple. The distance may be a k-mer distance.

We also apply our algorithms on data from previously published DNA-storage experiments and compare our accuracy and performance with state of the art algorithms known from the literature.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

In this work we present several reconstruction algorithms, crafted for DNA storage systems. Since our purpose is to solve the reconstruction problem as it is reflected in DNA-storage systems, our algorithms aim to minimize the distance between the output and the original strands. The algorithms in this work are different from most of the previously published reconstruction algorithms in several respects. Firstly, we do not require any assumption on the input. That is, the input can be arbitrary and does not necessarily belong to an error-correcting code. Secondly, our algorithms are not limited to specific cluster size, do not require any dependencies between the error probabilities, and do not assume zero errors in any specific location of the strands. Thirdly, we limit the complexity of our algorithms, so they can run with practical time on actual data from previous DNA-storage experiments. Lastly, since clusters in DNA storage systems may vary in their size and errors distributions, our algorithms are designed to minimize the distance between our output and the original strand, taking into account these errors can be corrected by the use of an error-correcting code.

We denote by Σ_{q}={0, . . . , q−1} the alphabet of size q and Σ_{q}* [symbol] [symbol] [symbol] . The length of x∈Σⁿ is denoted by |x|=n. The Levenshtein distance between two strings x, y∈Σ_{q}*, denoted by d_{L}(x, y), is the minimum number of insertions and deletions required to transform x into y. The edit distance between two strings x, y∈Σ_{q}*, denoted by dₑ(x,y), is the minimum number of insertions, deletions and substitution required to transform x into y, and d_{H}(x, y) denotes the Hamming distance between x and y, when |x|=|y|. For a positive integer n, the set {1, . . . , n} is denoted by [n].

### 0.4 The DNA Reconstruction Problem

The trace reconstruction problem was studied in several theoretical works. Under this framework, a length-n string x, yields a collection of noisy copies, also called traces, y₁, y₂, . . . , yₜ where each yᵢ is independently obtained from x by passing through a deletion channel, under which each symbol is independently deleted with some fixed probability p_{d}. Suppose the input string x is arbitrary. In the trace reconstruction problem, the main goal is to determine the required minimum number of i.i.d traces in order to reconstruct x with high probability. This problem has two variants: in the “worst case” , the success probability refers to all possible strings, and in the “average case” (or “random case”) the success probability is guaranteed for an input string x which is chosen uniformly at random.

The trace reconstruction problem can be extended to the model where each trace is a result of x passing through a deletion-insertion-substitution channel. Here, in addition to deletions, each symbol can be switched with some substitution probability pₛ, and for each j, with probability pᵢ, a symbol is inserted before the j-th symbol of x¹. Under this setup, the goal is again to find the minimum number of channels which guarantee successful reconstruction of x with high probability. ¹ Note that there are many interpretations for the deletion-insertion-substitution, most of them differ on the event when more than one error occured on the same index. Our interpretation of this channel is described in Section 0.14

Motivated by the storage channel of DNA and in particular the fact that different clusters can be of different sizes, this work is focused on another variation of the trace reconstruction problem, which is referred by the DNA reconstruction problem. The setup is similar to the trace reconstruction problem. A length-n string x is transmitted t times over the deletion-insertion-substitution channel and generates t traces y₁, y₂, . . . , yₜ. A DNA reconstruction algorithm is a mapping R: (Σ_{q}*)^{t}→Σ_{q}* which receives the t traces y₁, y₂, . . . , yₜ as an input and produces \hat{x}, an estimation of x. The goal in the DNA reconstruction problem is to minimize dₑ(x, \hat{x}), i.e., the edit distance between the original string and the algorithm's estimation. When the channel of the problem is the deletion channel, the problem is referred by the deletion DNA reconstruction problem and the goal is to minimize d_{L}(x, \hat{x}). While the main figure of merit in these two problems is the edit/Levenshtein distance, we will also be concerned with the complexity, that is, the running time of the proposed algorithms.

his section reviews the related works on the different reconstruction problems. In particular we list the reconstruction algorithms that have been used in previous DNA storage experiments and summarize some of the main theoretical results on the trace reconstruction problem.

### 0.5 Reconstruction Algorithms for DNA-Storage Systems

1. Baku et al. studied the trace reconstruction problem as an abstraction and a simplification of the multiple sequence alignment problem in bioinformatics. Here the goal is to reconstruct the DNA of a common ancestor of several organisms using the genetic sequences of those organisms. They focused on the deletion case of this problem and suggested a majority-based algorithm to reconstruct the sequence, which they referred by the bitwise majority alignment (BMA) algorithm. They aligned all traces by considering the majority vote per symbol from all traces, while maintaining pointers for each of the traces. If a certain symbol from one (or more) of the traces does not agree with the majority symbol, its pointer is not incremented and it is considered as a deletion. They showed and proved that even though this technique works locally for each symbol, its success probability is relatively high when the deletion probability is small enough.
2. Viswanathan and Swaminathan presented in 2008 a BMA-based algorithm for the trace reconstruction problem under the deletion-insertion-substitution channel. Their algorithm extends the BMA algorithm so it can support also insertions and substitutions. It works iteratively on “segments” from the traces, where a segment consists of consecutive bits and its size is a fixed fraction of the trace that is given as a parameter to the algorithm. The segment of each trace is defined by its pointers. The pointers of the traces are updated in each iteration similarly as in the BMA algorithm. Each trace is classified as valid or invalid by its distance from the majority segment. Once less than ¾ of the traces' segments are valid, the rest of the bits are estimated by the valid traces. Their algorithm extends and improves the results from Kannan et al., where it was shown that when the number of traces is O(log n) and the deletion/insertion probability is

$$O ( \frac{1}{{log}^{2} n} ) ,$$

it is possible to reconstruct a sequence with high probability. However, it assumes the deletion and insertion probabilities are relatively small, while the substitution probability is relatively large. In practice, these probabilities vary from cluster to cluster and do not necessarily meet these assumptions.
3. Gopalan et al. used the approach of the BMA algorithm and extended it to work with deletions, insertions, and substitutions to support DNA storage systems. They also considered a majority vote per symbol with some improvements. For any trace that its current symbol did not match the majority symbol, they used a “lookahead window” to look on the next 2 (or more) symbols. Then, they compared the next symbols to the majority symbols and classified it as an error accordingly. Organick et al. conducted a large scale DNA storage experiments where they successfully reconstructed their sequences using the reconstruction algorithm of Gopalan et al.
4. For the case of sequencing via nanopore technology, Duda et al. studied the trace reconstruction problem, while considering insertions, deletions, and substitutions. They focused on dividing the sequence into homopolymers (consecutive replicas of the same symbol), and proved that the number of copies required for accurately reconstructing a long strand is logarithmic with the strand's length. Yazdi et al. used a similar but different approach in their DNA storage experiment. They first aligned all the strands in the cluster using the multiple sequence alignment algorithm MUSCLE. Then, they divided each strand into homopolymers and performed majority vote to determine the length of each homopolymer separately. Their strands were designed to be balanced in their GC-content, which means that 50% of the symbols in each strands were G or C. Hence, they could perform additional majority iterations on the homopolymers' lengths until the majority sequence was balanced in its GC-content. All of these properties guaranteed successful reconstruction of the strands and therefore they did not need to use any error-correcting code in their experiment.

### 0.6 Theoreitical Results on the Trace Reconstruction Problem

1. Holenstein et al. presented an algorithm for reconstructing a random string using polynomially many traces from the deletion channel. In their work they assumed that the deletion probability is constant and is smaller than some threshold γ. Their work suggested a slightly different technique than the BMA technique, in the sense that they did not use standard majority voting, but a different majority scheme, where each trace vote is utilized with a probability measure of its certainty. They assumed that the last O(log n) of the input string (“anchor”) can not be affected by the deletion channel, or be removed to another part of the strings. Thus, the certainty of each trace is estimated by checking if the last O(log n) bits of the trace (“anchor”) match the last O(log n) of the recovered string. The threshold γ from was later estimated to be at most 0.07 by Peres and Zhai.
2. Peres and Zhai also improved the work by Holenstein et al. and the work by McGregor et al. They not only extended the range of the supported deletion probability to be [0, ½), but also showed that a subpolynomial number of traces, more specifically exp(O(log^{1/2} n)), is sufficient for the reconstruction of a random string. Their approach includes two steps, where in the first one the strings are aligned and then, in the second step, a majority-based algorithm is invoked in order to reconstruct the sequence. The alignment step of their algorithm is quite similar to the “anchor” technique as presented by Holenstein et al. The difference is that Peres and Zhai did not restrict the “anchor” to be just the last O(log n) bits in the strings, but it could be placed at any position in the traces.
3. Holden, Pemantle, and Peres improved the upper bound on the number of traces for the random case to (exp O(log^{1/3} n)) while both insertions and deletions are allowed in any probability from the range [0, 1). Their algorithm consists of three ingredients: (i) A boolean test T(w, w′) works on pairs of sequences of the same length and indicates whether w′ is likely to be a result of x passing through the deletion-insertion channel. (ii) An alignment procedure that creates for each of the traces yᵢ an estimate τ for the matching between positions in yᵢ and x. (iii) A bit recovery procedure that uses the aligned traces in order to estimate a bit or subsequence of bits.
4. For the worst case scenario, Holenstein et al. proved that exp O(n^{1/2} log n) traces suffice for reconstruction with high probability. This was later improved independently by both De, O'Donnell, and Severdio and by Nazarov and Peres to exp O(n^{1/3}).
5. Two recent works studied the trace reconstruction problem for the codes setup, i.e., the transmitted is sequence not arbitrary but belongs to some code with error-correction capabilities.
6. Another related model has been studied by Kiah et al. who introduced another approach for the trace reconstruction problem, where they used profile-vectors-based coding scheme in order to reconstruct the sequence.
7. Another related problem, phrased as the sequence reconstruction problem, was also studied by Levenshtein, but his approach was different. Under this paradigm he studied the minimum number of different (noisy) channels that is required in order to build a decoder that can reconstruct any transmitted sequence in the worst case. Levenshtein showed that the number of channels that is required to recover any sequence has to be greater than the maximum intersection between the error balls of any two transmitted sequences. Since Levenshtein studied the worst case of this problem, the number of unique channels has to be extremely large which is not applicable for the practical setup we consider in the reconstruction of DNA strands in a DNA-based storage system.

While all previous works of reconstructing algorithms used variations of the majority algorithm on localized areas of the traces, we take a different global approach to tackle this problem. Namely, the algorithms presented in the paper are heavily based on the maximum likelihood decoder for multiple deletion channels as well as the concepts of the shortest common supersequence and the longest common subsequence.

### 0.7 Supersequences and Subsequences

For a sequence x∈E_{q}* and a set of indices I⊆[|x|], the sequence x_{I} is the projection of x on the indices of I which is the subsequence of x received by the symbols at the entries of I. A sequence x∈Σ* is called a supersequence of y∈Σ*, if y can be obtained by deleting symbols from x, that is, there exists a set of indices I⊆[|x|] such that y=x_{I}. In this case, it is also said that y is a subsequence of x. Furthermore, x is called a common supersequence (subsequence) of some sequences y₁, . . . , yₜ if x is a supersequence (subsequence) of each one of these t sequences. The length of the shortest common supersequence (SCS) of y₁, . . . , yₜ is denoted by SCS(y₁, . . . , yₜ). The set of all shortest common supersequences of y₁, . . . , yₜ∈Σ* is denoted by [symbol] CS(y₁, . . . , yₜ). Similarly, the length of the longest common subsequence (LCS) of y₁, . . . , yₜ, is denoted by LCS(y₁, . . . , yₜ), and the set of all longest subsequences of y₁, . . . , yₜ is denoted by [symbol] CS(y₁, . . . , yₜ).

### 0.8 Maximum Likelihood Decoder for Multiple Deletion Channels

Consider a channel S that is characterized by a conditional probability Pr_{S}, which is defined by

Pr_{S}{y rec. |x trans.},

for every pair (x, y)∈(Σ_{q}*)². Note that it is not assumed that the lengths of the input and output sequences are the same as we consider also deletions and insertions of symbols. As an example, it is well known that if S is the binary symmetric channel (BSC) with crossover probability 0 [symbol] p [symbol] ½, denoted by BSC(p), it holds that Pr_{BSC(p)}{y rec. |x trans.}=p^{d}^{_{H}}^{(y,x)}(1−p)^{n-d}^{_{H}}^{(y,x)}, for all (x, y)∈(Σ₂ⁿ)², and otherwise (the lengths of x and y is not the same) this probability equals 0.

The maximum-likelihood (ML) decoder for a code C with respect to S, denoted by [symbol] _{ML}, outputs a codeword c∈C that maximizes the probability Pr_{S}{y rec. |c trans.}. That is, for y∈Σ_{q}*,

$${𝒟}_{M L} ( y ) = {argmax}_{c ∈ 𝒞} \{ {Pr}_{S} \{ y rec . \vert c trans . \} \} .$$

It is well known that for the BSC, the ML decoder simply chooses the closest codeword with respect to the Hamming distance.

The conventional setup of channel transmission is extended to the case of more than a single instance of the channel. Assume a sequence x is transmitted over some t identical channels of S and the decoder receives all channel outputs y₁, . . . , yₜ. This setup is characterized by the conditional probability

$$P {r}_{( S , t )} \{ {y}_{1} , … , {y}_{t} re c . \vert x trans . \} = {\prod }_{i = 1}^{t} P {r}_{S} \{ {y}_{i} re c . \vert x trans . \} .$$

Now, the input to the ML decoder is the sequences y₁, . . . , yₜ and the output is the codeword c which maximizes the probability Pr_{(S,t)}{y₁, . . . , yₜ rec.|trans.}.

For two sequences x, y∈Σ_{q}*, the number of times that y can be received as a subsequence of x is called the embedding number of y in x and is defined by

Emb(x; y)=|{I⊆[|x|]|x_{I}=y}∥.

Note that if y is not a subsequence of x then Emb(x; y)=0. The embedding number has been studied in several previous works and was referred to as the binomial coefficient. In particular, this value can be computed with quadratic complexity.

While the calculation of the conditional probability Pr_{S}{y rec. |x trans.} is a rather simple task for many of the known channels, it is not straightforward for channels which introduce insertions and deletions. In the deletion channel with deletion probability p, denoted by Del(p), every symbol of the word x is deleted with probability p. For the deletion channel it is known that for all (x, y)∈(Σ_{q}*)², it holds that

Pr_{Del(p)}{y rec. |x trans.}=p^{|x|-|y|}·(1−p)^{|y|}·Emb(x; y).

According to this property, the ML decoder for one or multiple deletion channels is stated as follows:.
Lemma 1. Assume c∈C⊆(Σ_{q})ⁿ is the transmitted sequence and y₁, . . . , yₜ∈(Σ_{q})* are the output sequences from Del(p), then

$${𝒟}_{M L} ( {y}_{1} , … , {y}_{t} ) = {argmax}_{x ∈ CS ( {y}_{1} , … , {y}_{t} )} \{ {\prod }_{i = 1}^{t} E m b ( x ; {y}_{i} ) \cdot {( 1 - p )}^{\vert {y}_{i} \vert} \cdot {p}^{\vert x \vert - \vert {y}_{i} \vert} \} .$$

Note that since there is more than a single channel, when the goal is to minimize the average decoding error probability, the ML decoder does not necessarily have to output a codeword but any sequence that minimizes the average decoding error probability. In the next sections it will be shown how to use the concepts of the SCS and LCS together with the maximum likelihood decoder in order to build decoding algorithms for the deletion DNA reconstruction and the DNA reconstruction problems.

This section studies the deletion DNA reconstruction problem. Assume that a cluster consists of t traces, y₁, y₂, . . . , yₜ, where all of them are noisy copies of a synthesized strand. This model assumes that every strand is a sequence that is independently received by the transmission of a length-n sequence x (the synthesized strand) through a deletion channel with some fixed deletion probability p_{d}. Our goal is to propose an efficient algorithm which returns \hat{x}, an estimation of the transmitted sequence x, with the intention of minimizing the Levenshtein distance between x and \hat{x}. We consider both cases when t is a fixed small number and large values of t.

### 0.9 An Algorithm for Small Fixed Values of t

Our approach is based on the maximum likelihood decoder over the deletion channel. A straightforward implementation of this approach on a cluster of size t is to compute the set of shortest common supersequences of y₁, y₂, . . . , yₜ, i.e., the set [symbol] CS(y₁, y₂, . . . , yₜ), and then return the maximum likelihood sequence among them. This algorithm has been rigorously studied to analyze its Levenshtein error rate for t=2. The method to calculate the length of the SCS commonly uses dynamic programming and its complexity is the product of the lengths of all sequences. Hence, even for moderate cluster sizes, e.g. t [symbol] 5, this solution will incur high complexity and impractical running times. However, for many practical values of n and p_{d}, the original sequence x can be found among the list of SCSs while taking less than t traces or even only two of them. This fact, which we verified empirically, can drastically reduce the complexity of the ML-based algorithm. Furthermore, note that x is always a common supersequence of all traces, however it is not necessarily the shortest one. Hence, our algorithm works as follows. The algorithm creates sorted sets of r-tuples, where each tuple consists of r traces from the cluster. The r-tuples are sorted in a non-decreasing order according to the sum of their lengths. For each r-tuple (yᵢ_{₁}, . . . , yᵢ_{ᵣ}), the algorithm first calculates its length of the SCS, i.e., the value SCS(yᵢ_{₁}, . . . , yᵢ_{ᵣ}). Observe that if SCS(yᵢ_{₁}, . . . , yᵢ_{ᵣ})=n then the sequence x necessarily appears in the set of SCSs of (yᵢ_{₁}, . . . , yᵢ_{ᵣ}), that is, x∈ [symbol] CS(yᵢ_{₁}, . . . , yᵢ_{ᵣ}). However it is not necessarily the only sequence in [symbol] CS(yᵢ_{₁}, . . . , yᵢ_{ᵣ}). Hence, all is left to do is to filter the set [symbol] CS(yᵢ_{₁}, . . . , yᵢ_{ᵣ}) with sequences that are supersequences of all t traces and finally return the maximum likelihood among them. The algorithm iterates over all possible r-tuples for r=2, 3, 4 and if none of them succeeds, the algorithm computes all SCSs of maximal length that were observed throughout its run and returns the one that minimizes the sum of Levenshtein distances from all copies in the cluster.

In Algorithm 1, we present a pseudo-code of our solution for the deletion DNA reconstruction problem. Note that the algorithm uses another procedure which is presented in Algorithm 2 to filter the supersequences and output the maximum likelihood supersequence. The input to the algorithm is the length n of the original sequence, and a cluster of t traces C. Algorithm 1's main loop is in Step 2; first in Step 2-a it generates the set F, which is a sorted set of all r-tuples of traces by the sum of their lengths. Then, in Step 2-b it iterates over all r-tuples in F and checks for each r-tuple, (yᵢ_{₁}, . . . , yᵢ_{ᵣ}), if the length of their SCS, i.e., SCS(yᵢ_{₁}, . . . , yᵢ_{ᵣ}), equals n. If it is equal to n, it computes the set of all its SCSs, [symbol] CS(yᵢ_{₁}, . . . , yᵢ_{ᵣ}), and invokes Algorithm 2. Algorithm 2 checks if one or more of those SCSs are supersequences of all of the traces in the cluster, and if so it returns the maximum likelihood among them. In case that SCS(yᵢ_{₁}, . . . , yᵢ_{ᵣ})<n, the algorithm checks also if it is equal or greater than nₘₐₓ, which is the longest SCS that was found so far. In this case, the algorithm saves Cₘₐₓ, which is the set of all r-tuples such that the length of their SCS equals nₘₐₓ. In Step 3, the algorithm computes Sₘₐₓ=∪_{c∈C}_{ₘₐₓ} [symbol] CS(c), which is the union of sets of SCSs of the r-tuples that the length of their SCS was nₘₐₓ. In Step 4, the algorithm invokes again Algorithm 2 to check if Sₘₐₓ includes supersequences of all traces in C and returns the maximum likelihood among them. If none of the sequences in Sₘₐₓ is a supersequence of all traces in C, the algorithm returns in Step 5 the sequence which minimizes the sum of Levenshtein distances to all the traces in C.

### 0.10 Simulations

We evaluated the accuracy and efficiency of Algorithm 1 by the following simulations. These simulations were tested over sequences of length n=200, clusters of size 4 [symbol] t [symbol] 10, and deletion probability p in the range [0.01, 0.10]. The alphabet size was 4. Each simulation consisted of 100,000 randomly generated clusters. Furthermore, we had another set of simulations for n=100 with deletion probability p in the range [0.11, 0.20] and clusters of size 4 [symbol] t [symbol] 10. Each simulation for these values of p,n, and t included 10,000 randomly selected clusters. We calculated the Levenshtein error rate (LER) of the decoded output sequence as well as the average decoding success probability (referred as the success rate). We also calculated the kerror success rate, which is defined as the fraction of clusters where the Levenshtein distance between the algorithm's output sequence and the original sequence was at most k. Note that for k=0, this is equivalent to calculate the success rate. We also calculated the minimal k for which its k-error success rate is at least q, and denote this value of k by k_{q_succ}. Note that for q=1 this value determines the minimal number of Levenshtein errors that an error-correcting code must correct in order to fully decode the original sequences using Algorithm 1 with an error-correcting code. In addition, each cluster was also reconstructed using the BMA algorithm.

Algorithm 1 ML-SCS Reconstruction

Input:

| | |
|---|---|
| • | Cluster C of t noisy traces: y₁,y₂,...,yₜ sorted by their lengths from the |
| | longest to the shortest. |
| • | Design length= n. |

Output: \hat{x} - Estimation of the original sequence.

| 1. | \hat{x} = ϵ, nₘₐₓ = 0; Cₘₐₓ = ∅. | | |
|---|---|---|---|
| 2. | for r = 2, 3, 4 do | | |
| | | (a) | Denote F={cᵢ^{(r)} = (yᵢ^{₁},yᵢ^{₂},...,yᵢ^{ᵣ})\|1 [symbol] i [symbol] (ᵣ^{t}),1 [symbol] i₁ < i₂ < |
| | | | ... < iᵣ [symbol] t} the set of all r-tuples from C, sorted by non-decreasing |
| | | | order of the sum of the lengths of the copies in each tuple. |
| | | (b) | for i = 1, 2, ..., (ᵣ^{t}) do |
| | | | if SCS(cᵢ^{(r)}) = n then |
| | | | S = SCS(cᵢ^{(r)}) |
| | | | \hat{x} = ML-Supersequence(S, C) |
| | | | if \hat{x} ≠ ϵ then |
| | | | return \hat{x} |
| | | | end if |
| | | | else |
| | | | if [symbol] CS(cᵢ^{(r)}) > nₘₐₓ then |
| | | | nₘₐₓ = SCS(cᵢ^{(r)}) |
| | | | Cₘₐₓ = {cᵢ^{(r)}} |
| | | | end if |
| | | | if SCS(cᵢ^{(r)}) = nₘₐₓ then |
| | | | Cₘₐₓ = Cₘₐₓ ∪ {cᵢ^{(r)}} |
| | | | end if |
| | | | end if |
| | | (c) | end for |

end for

3. Compute Sₘₐₓ = ∪_{c∈C}^{ₘₐₓ} [symbol] CS(c), the union of all [symbol] CS of cᵢ^{(r)} ∈Cₘₐₓ.
4. \hat{x} = ML-Supersequene(Sₘₐₓ,C)

| | |
|---|---|
| 5. | if \hat{x} ≠ ϵ then |
| | return \hat{x} |
| | else |
| | Return the sequence from Sₘₐₓ that has the minimum sum of Levenshtein distance to |
| | the copies in the cluster. |
| | end if |

Algorithm 2 ML-Supersequence

Input:

| | |
|---|---|
| • | Cluster C of t noisy traces: y₁,y₂,...,yₜ. |
| • | S={s₁,s₂,...,sₖ}, a set of k candidates. |

Output:
Maximum likelihood candidate of S, that is supersequence of all copies
from the cluster. If it is not exists, the algorithm returns ϵ.

| | |
|---|---|
| 1. | Filter S so it contains only sequences which are supersequence |
| | of all traces from the cluster. |
| 2. | if S ≠ ∅ then |
| | Return the maximum likelihood sequence from S with a |
| | respect to cluster C. |
| | end if |
| 3. | Return ϵ. |

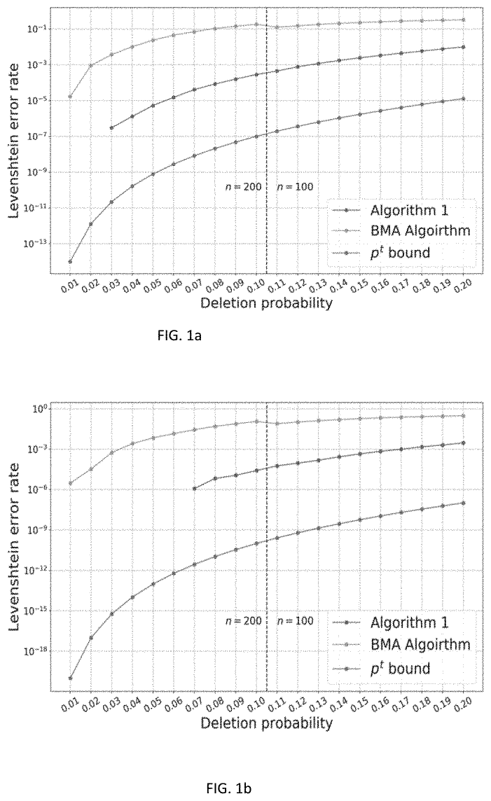

FIG. 1 presents the LER as computed in our simulations of Algorithm 1 and the BMA algorithm for clusters of sizes t=7 and t=10. We also added the trivial lower bound of p^{t} on the LER. This bound corresponds to the case when the same symbol is deleted in all of the traces. In this case, this symbol will not appear in the list of SCSs of any possible r-tuple or even the entire cluster since it cannot be recovered. Hence, it is not possible to recover its value and thus it will be deleted also in the output of the ML decoder.

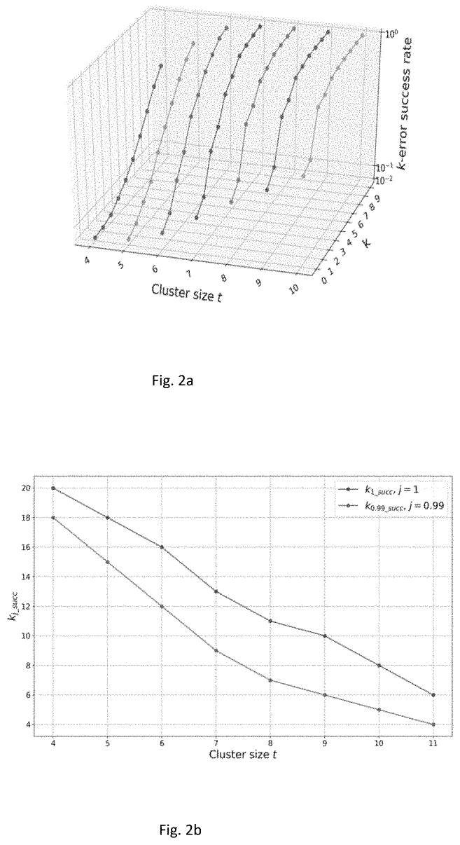

In order to simulate also high deletion probabilities, we simulated 1000 clusters of sequences over 4-ry alphabet of length n=100 with cluster size t between 4 and 10, while the deletion probability was p=0.25. FIG. 2(a) presents the k-error success rate of this simulation and FIG. 2(b) presents the values of k_{1_succ} and k_{0.99.succ} by the cluster size in the simulation.

### 0.11 Large Cluster

In case the cluster is of larger size, for example in the order of Θ(n), we present in Algorithm 3, a variation of Algorithm 1 for large clusters. In this case, since the cluster is large, the probability to find a pair, triplet, or quadruplet of traces that their set of SCSs contains the original sequence x is very high, if not even 1. In fact, in all of our simulations, which we will elaborate below in this section, we were always able to successfully decode the original sequence with no errors even when the deletion probability was as high as 0.2. Hence, our main goal in this part is to decrease the runtime of Algorithm 1 while preserving the success rate to be 1. Algorithm 3 keeps the same structure of Algorithm 1, however, it performs two filters on the cluster in order to reduce the computation time.

The complexity of finding the length of the SCS of some set of r traces is the multiplication of their lengths, i.e., Θ(n^{r}). Therefore, the complexity of finding the length of the SCS of a pair of traces is Θ(n²), while there are Θ(n²) pairs of traces (assuming the cluster size is Θ(n)). Therefore, in this case, calculating the length of the SCS of each pair of traces before considering some triplets is not necessarily the right strategy when our goal is to optimize the algorithm's running time. Hence, in Algorithm 3 we focused on filtering the traces in the cluster in order to check only a subset of the traces which are more likely to succeed and produce the correct sequence.

To define the filtering criteria for Algorithm 3, we simulated Algorithm 1 on large clusters. The length of the original sequence x was n=200 and the cluster size was

$$t = \frac{n}{2} = 100 .$$

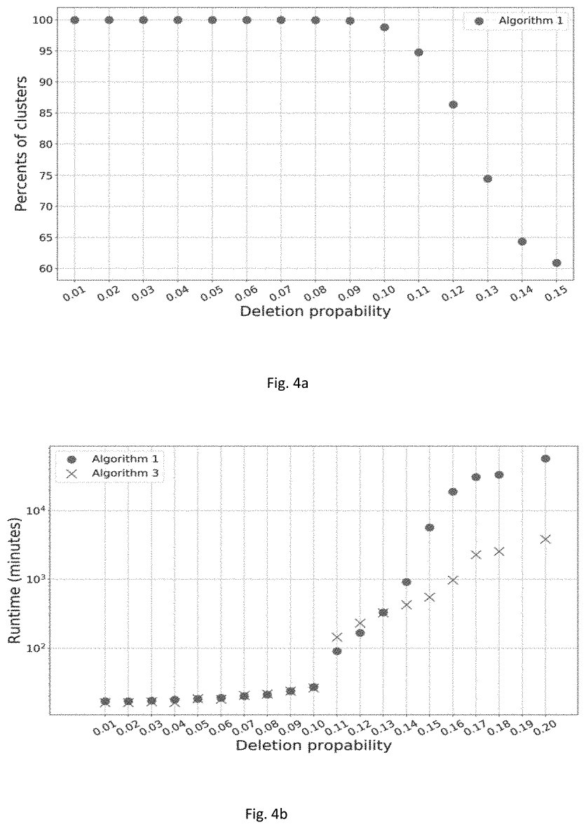

We generated 10,000 clusters of size t, where the deletion probability p was in the range [0.01, 0.15]. The success rate of all the simulations was 1. We evaluated the percentage of clusters that the first r-tuple to have an SCS of length n was consisted of the longest 20% traces in the cluster. We observed that when the deletion probability was at most 0.07, in all of the clusters the first r-tuple of traces that had an SCS of size n consisted from the longest 20% traces in the cluster. For deletion probabilities between 0.08 and 0.11 these percentages ranged between 94.76% and 99.98%, while for p=0.15 this percentage was 60.88%. Therefore, by filtering the longest 20% traces, it was enough to check only (₂²⁰) pairs instead of (₂¹⁰⁰) (pairs in order to succeed and still reach the successful pair. The results of these simulations are depicted in FIG. 4(a).

This observation lead us to the first filter in Algorithm 3, where we picked the longest 20% traces of the cluster. The second filter computes a cost function (in linear time complexity), to be explained below, on a given r-tuple of traces in order to evaluate if the traces in this r-tuple are likely to have an SCS of length n. Thus, the algorithm skips on the SCS computation of r-tuples that are less likely to have an SCS of length n. First, before performing the first filter, the algorithm calculates the average length of the traces in the cluster and uses it to estimate the deletion probability p. Then, if p>0.1, the algorithm calculates the cost function on every r-tuple and checks if it is higher than some fixed threshold. This threshold depends on the estimated value of p and the cost function is based on a characterization of the sequences, as will be described in Section 011.2.

### 0.11.1 An Algorithm for Large Values of t

In this section we present Algorithm 3. We list here the steps that are different from Algorithm 1. In Step 2 the algorithm estimates the deletion probability in the cluster by checking the average length of the traces n′ and then calculates

$$p = 1 - \frac{{n}^{′}}{n} .$$

In Step 3, the algorithm filters the cluster so it contains only the longest 20% traces. The last difference between Algorithm 3 and Algorithm 1 can be found in Step 4-b. In this step, before the computation of the SCS of a given r-tuple of traces, the algorithm computes the k-mer cost function (for k-mers of size k=2) and checks if it is larger than the threshold Tₚ.

We evaluated the performance of Algorithm 3 and verified our filters by simulations. Each simulation consisted of 10,000 clusters of size t=100, the length of the original strand was n=200, the alphabet size was q=4, and the deletion probability p was in the range [0.01, 0.2]. Algorithm 3 reconstructed the exact sequence x in all of the tested clusters. A comparison between the runtime of Algorithm 1 and Algorithm 3 can be found in FIG. 4(b). Note that we did not compare the running time with the BMA algorithm since its success rate was significantly lower, for example when the deletion probability was 15%, its success rate was roughly 0.46.

### 0.11.2 The k-Mer Distance and the K-Mer Cost Function

The k-mer vector of a sequence y, denoted by k-mer(y), is a vector that counts the frequency in y of each subsequence of length k (k-mer). The frequencies are ordered in a lexicographical order of their corresponding k-mers. For example for a given sequence y=“ACCTCC” and k=2, its k-mer vector is k-mer(y)=0100020100000101, according to the following calculation of the frequencies {AA:0, AC:1, AG:0, AT:0, CA:0,CC:2,CG:0,CT:1,GA:0,GC:0,GG:0,GT:0,TA:0,TC:1,TG:0,TT:1}. We define the k-mer distance between two sequences y₁ and y₂ as the L₁ distance between their k-mer vectors. The k-mer distance is denoted by dₖ₋ₘₑᵣ(y₁, y₂)

dₖ₋ₘₑᵣ(y₁,y₂)=∥y₁−y₂∥₁

. For a given set of r sequences Y={y₁, y₂, . . . yᵣ}, we define its k-mer cost function, which is denoted by cₖ₋ₘₑᵣ(y₁, y₂, . . . , yᵣ), as the sum of the k-mer distance of each pair of sequences in Y. That is,

$${c}_{k ‐ mer} ( {y}_{1} , {y}_{2} , … , {y}_{r} ) = {\sum }_{1 ⩽ i < j ⩽ r} {d}_{k ‐ mer} ( {y}_{i} , {y}_{j} ) .$$

Observe that the k-mer distance between a sequence x and a trace y₁ which results from x by one deletion is at most 2k−1. Every deleted symbol in x decreases the

Algorithm 3 ML-SCS Reconstruction for Large Clusters

Input:

| | |
|---|---|
| • | Cluster C of t = Θ(n) noisy traces: y₁, y₂, . . . , yₜ sorted by their lengths in a |
| | non-decreasing order. |
| • | Design length= n. |

Output: \hat{x} - Estimation of the original sequence.

| 1. | \hat{x} = ϵ, nₘₐₓ = 0, Cₘₐₓ = Ø. | |
|---|---|---|
| 2. | $Compute {n}^{′} the mean length of the traces in C , and define p = 1 - \frac{{n}^{′}}{n} .$ | |
| 3. | Filter traces from C so it contains only the t' = 0.2t first traces in the cluster. | |
| 4. | for r = 2, 3, 4 do | |
| | (a) | $Denote F = \{ {c}_{i}^{( r )} = ( {y}_{{i}_{1}} , {y}_{{i}_{2}} , … , {y}_{{i}_{r}} ) \vert 1 ⩽ i ⩽ ( \begin{matrix}{t}^{′} \\ r\end{matrix} ) , 1 ⩽ {i}_{1} < {i}_{2} <$ |
| | | . . . < iᵣ [symbol] t'} the set of all r-tuples from C, sorted by non-decreasing |
| | | order of the sum of the lengths of the copies in each tuple. |
| | (b) | $for i = 1 , 2 , … , ( \begin{matrix}{t}^{′} \\ r\end{matrix} ) do$ |
| | | if p > 0.1 and cₖ₋ₘₑᵣ(cᵢ^{(r)}) > 0.25np(2k − 1) then |
| | | /* k-mer size k = 2. */ |
| | | if SCS(cᵢ^{(r)}) = n then |
| | | S = [symbol] CS(cᵢ^{(r)}) |
| | | \hat{x}= ML-Supersequence(S, C) |
| | | if \hat{x} ≠ ϵ then |
| | | return \hat{x} |
| | | end if |
| | | else |
| | | if SCS(cᵢ^{(r)}) > nₘₐₓ then |
| | | nₘₐₓ = SCS(cᵢ^{(r)}) |
| | | Cₘₐₓ = Cₘₐₓ ∪ {cᵢ^{(r)}} |
| | | end if |
| | | if SCS(cᵢ^{(r)}) = nₘₐₓ then |
| | | Cₘₐₓ = Cₘₐₓ ∪ {cᵢ^{(r)}} |
| | | end if |
| | | end if |
| | | end if |
| | (c) | end for |

end for

5. Compute Sₘₐₓ=∪_{cϵC}_{ₘₐₓ} SCS(c), the union of all [symbol] CS of cᵢ^{(r)} ϵC_{ₘₐₓ}.
6. \hat{x}=ML-Supersequence(Sₘₐₓ, C)

| | |
|---|---|
| 7. | if \hat{x} ≠ ϵ then |
| | return \hat{x} |
| | else |
| | Return the sequence from Sₘₐₓ that has the minimum sum of Levenshtein distance to |
| | the copies in the cluster. |
| | end if |

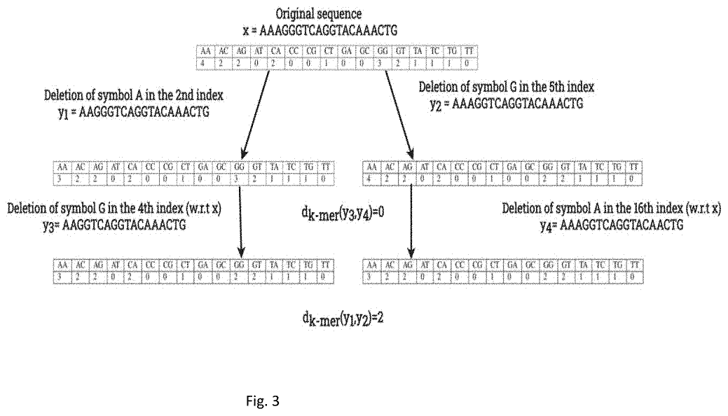

value of at most k entries in k-mer(x) and increases the number of at most k−1 of the entries. Hence, each deletion increases the k-mer distance by at most 2k−1, which means that an upper bound on the k-mer distance between the original strand x and a trace yᵢ with np deletions is np(2k−1). However, when comparing the k-mer distance of two traces, y₁ and y₂, with more than one deletion, the k-mer distance can also decrease. An example of such a case is depicted in FIG. 3. Combining these two observations, Algorithm 3 estimates if two traces have relatively large Levenshtein distance. If these traces have large Levenshtein distance, it is more likely that both of them will have an SCS of length n. Hence, the algorithm checks if the k-mer distance is larger than the threshold Tₚ=0.25np(2k−1) and continues to compute the SCS, only if the condition holds. A similar computation is done for tuples with more than two traces. We use the value of 0.25 in the threshold to consider the cases where the k-mer distance decreases as depicted in FIG. 3. We selected this value after simulating other values as well, reaching the best result with 0.25. An optimization of this value can be done in further research.

This section studies the DNA reconstruction problem. Assume that a cluster consists of t traces, y₁, y₂, . . . , yₜ, where all of them are noisy copies of a synthesized strand. This model assumes that every trace is a sequence that is independently received by the transmission of a length-n sequence x (the synthesized strand) through a deletion-insertion-substitution channel with some fixed probability p_{d} for deletion, pᵢ for insertion, and pₛ, for substitution. Our goal is to propose an efficient algorithm which returns \hat{x}, an estimation of the transmitted sequence x, with the intention of minimizing the edit distance between x and \hat{x}. In our simulations, we consider several values of t and a wide range of error probabilities as well as data from previous DNA storage experiments.

Before we present the algorithms, we list here several more notations and definitions. An error vector of y and x, denoted by EV(y, x), is a vector of minimum number of edit operations to transform y to x. Each entry in EV(y, x) consists of the index in y, the original symbol in this index, the edit operation and in case the operation is an insertion, substitution the entry also includes the inserted, substituted symbol, respectively. Note that for two sequences y and x, there could be more than one sequence of edit operations to transform y to x. The edit distance between a pair of sequences is computed using a dynamic programming table and the error vector is computed by backtracking on this table. Hence, EV(y, x) is not unique and can be defined uniquely by giving priorities to the different operation in case of ambiguity. That is, if there is an entry in the vector EV(y, x) (from the last entry to the first), where more than one edit operation can be selected, then, the operation is selected according to these given priorities. The error vector EV(y, x) also maps each symbol in y to a symbol in x (and vice versa). We denote this mapping as V_{EV}(y, x): {1, 2, . . . , |y|}→{1, 2, . . . , |x|}∪{?}, where V_{EV}(y, x)(i)=j if and only if the i-th symbol in y appears as the j-th symbol in x, with respect to the error vector EV(y, x). Note that in the case where the i-th symbol in y was classified as a deleted symbol in EV(y, x), V_{EV}(y, x)(i)=?. This mapping can also be represented as a vector of size |y|, where the i-th entry in this vector is V_{EV}(y, x)(i). The reversed cluster of a cluster C, denoted by C^{R}, consists of the traces in C where each one of them is reversed.

### 0.12 The LCS-Anchor Algorithm

In this section we present Algorithm 4, the LCS-anchor algorithm. The algorithm receives C, a cluster of traces sorted by their lengths, from closest to n to the farthest to n. First, the algorithm initializes \hat{x} and \hat{x}^{bck} as a length-n sequence of the symbol ‘-’. Second, the algorithm computes lcs, an arbitary LCS of y₁ and y₂, the two traces in the cluster which their length is closest to n. Then, for each of the t traces in the cluster, yₖ, the algorithm computes EV(yₖ, lcs), and the mapping vector V_{EV}(lcs, yₖ). For 1 [symbol] i [symbol] |lcs| the algorithm performs a majority vote on the i-th entries of the t mapping vectors. If the majority is j≠?, the algorithm writes the symbol lcs(i) in the j-th index of \hat{x}. If j=? the symbol lcs(i) is omitted, and it is not written in \hat{x}. At this point, Algorithm 4 wrote into \hat{x} at most LCS(y₁, y₂) symbols, these symbol serves as “anchor” symbols in the estimated string. Each of the anchor symbols is located in a specific index of \hat{x}, and the rest of the symbols in \hat{x} are ‘-’. Note that the anchor symbols are not necessarily placed in consecutive indices of \hat{x}. In the following steps, Algorithm 4 computes for all yₖ∈C, the vectors EV(\hat{x}, yₖ) and V_{EV}(\hat{x}, yₖ) Then, for each h, an entry of ‘-’ in \hat{x}, the algorithm performs a majority vote on yₖ(V_{EV}(\hat{x}, yₖ)(h)), to find the most frequent symbol in this entry, and saves it in the h-th entry of \hat{x}. Lastly, the algorithm performs these steps on C^{R} and saves the resulted sequence in \hat{x}^{bck}. Algorithm 4 returns as output

$${\hat{x}}_{1 , 2 , … , ⌈ \frac{n}{2} ⌉} {\hat{x}}_{1 , … , ⌊ \frac{n}{2} ⌋}^{b c k} .$$

### 0.13 The Iterative Reconstruction Algorithm

In this section we present Algorithm 5. The algorithm receives a cluster of t traces C and the design length n. Algorithm 5 uses several methods to revise the traces from the cluster and to generate from the revised traces a multiset of candidates. Then, Algorithm 5 returns the candidate that is most likely to be the original sequence x. The methods used to revise the traces are described in this section as Algorithm 6 and Algorithm 7. Algorithm 5 invokes Algorithm 6 and Algorithm 7 on the cluster in two different procedures as described in Algorithm 8 and Algorithm 9.

The first method is described in Algorithm 6. The algorithm receives C, a cluster oft traces, the design length n, and yᵢ, a trace from the cluster. Algorithm 6 calculates for every 1 [symbol] k [symbol] t, k≠i, the vector EV(yᵢ, yₖ). In some of the cases, there may be more than one error vector for EV(yᵢ, yₖ), which corresponds to the edit operations to transform yᵢ to yₖ. In these cases, the algorithm prioritizes substitutions, then insertions, then deletions in order to choose one unique vector². Then, the algorithm performs a majority vote in each index on these vectors and creates S, which is a vector of edit operations. Lastly, Algorithm 6 performs the edit operations on yᵢ, and returns it as an output for Algorithm 8 and Algorithm 9. Algorithm 6 is used as a procedure in Algorithm 8 and Algorithm 9 to correct substitution and insertion errors of the traces in the cluster. ²These priorities were selected to support our definition of the deletion-insertion-substitution channel. However, for practical uses, one can easily change these priorities if some preliminary knowledge of the error rates in the data is given.

The second method is described in Algorithm 7. Similarly to Algorithm 6, Algorithm 7 receives C, a cluster of t traces, the design length n, and yₖ, a trace from the cluster. Algorithm 7 uses similar patterns (defined in Section 0.13.1) on each pair of traces and creates a weighted graph from them. Each vertex of the graph represents a pattern, and an edge connects patterns with identical prefix and suffix. The weight on each edge represents the frequency of the incoming pattern,

Algorithm 4 LCS-Anchor

Input:

| • | Cluster C of t noisy traces: y₁, y₂, . . . , yₜ, sorted by their lengths | |
|---|---|---|
| | from closest to the farthest to n. | |
| • | Design length = n | |
| | Output: | |
| • | \hat{x} - Estimation of the original sequence | |
| 1. | Initialize \hat{x} and \hat{x}^{bck} as a length-n sequence of the symbol ‘−’. | |
| 2. | Calculate lcs one of the LCSs of y₁, y₂. | |
| 3. | for all yₖ ∈ C do | |
| | (a) | Compute EV(yₖ, lcs). |
| | (b) | Compute V_{EV}(lcs, yₖ). |
| | end for | |
| 4. | for 1 [symbol] i [symbol] lcs do | |
| | (a) | j = 0 |
| | (b) | For 1 [symbol] k [symbol] t, perform a majority vote on V_{EV}(lcs, yₖ)(i) |
| | | and save it in j. |
| | (c) | If j ≠?, \hat{x}(j) = lcs(i) |
| | end for | |
| 5. | for all yₖ ∈ C do | |
| | (a) | Compute EV(yₖ, \hat{x}). |
| | (b) | Compute V_{EV}(\hat{x}, yₖ). |
| | end for | |
| 6. | for all h s.t \hat{x}(h) =‘−’ do | |
| | (a) | For 1 [symbol] k [symbol] t, perform a majority vote on yₖ(V_{EV}(\hat{x}, yₖ)(h)) |
| | | and save it in m. |
| | (b) | \hat{x}(h) = m. |

| | |
|---|---|
| | end for |
| 7. | Repeat Steps 2 - 6 for C^{R} and save the results in \hat{x}^{bck}. |
| 8. | $Return {\hat{x}}_{1 , 2 , … , ⌈ \frac{n}{2} ⌉} {\hat{x}}_{1 , 2 , … , ⌊ \frac{n}{2} ⌋}^{bck}$ |

the number of pairs of traces in the cluster that have this pattern in their sequences. Algorithm 7 is described in detail in Section 9A.3.1. Algorithm 7 is used as a procedure in in Algorithm 8 and Algorithm 9 to correct deletion errors in the traces in the cluster.

Algorithm 8 receives a cluster of t traces C and the design length n. Algorithm 8 performs k cycles, where in each cycle it iterates over all the traces in the cluster. For each trace yₖ, it first uses Algorithm 6 to correct substitution errors, then it uses Algorithm 7 to correct deletion errors, and lastly, it uses Algorithm 6 to correct insertion errors. When it finishes iterating over the traces in the cluster, Algorithm 8 updates the cluster with all the revised traces and continues to the next cycle. At the end, Algorithm 8 performs the same procedure on C^{R}. Algorithm 8 returns a multiset of all the revised traces.

Algorithm 9 also receives a cluster of t traces C and the design length n. Algorithm 9 uses the same procedures as Algorithm 8. However, in each cycle, it first corrects substitutions in all of the traces in the cluster using algorithm 6, then it invokes algorithm 7 on each trace to correct deletions, and finally invokes Algorithm 6 to correct insertions. Similarly to Algorithm 8, Algorithm 9 performs the same operations also on C^{R} and returns a multiset of the results.

Algorithm 5 invokes Algorithms 8 and 9, with k=2 cycles and combines the resulted multisets to the multiset S. If one or more sequences of length n exists in the multiset S, it returns the one that minimizes the sum of edit distances to the traces in the cluster. Otherwise, it checks if there are sequences of length n−1 or n+1 in S, and returns the most frequent among them. If such a sequence does not exists, it returns the first sequence in S.

### 0.13.1 The Pattern Path Algorithm

In this section we present Algorithm 7, the Pattern-Path algorithm. Algorithm 7 is being used to correct deletion errors. Denote by w an arbitrary LCS sequence of x and y of length [symbol] . The sequence w is a subsequence of x, and hence, all of its [symbol] symbols appear in some indices of x³, and assume these indices are given by

Algorithm 5 Iterative Reconstruction

Input:

| | |
|---|---|
| • | Cluster C of t noisy traces: y₁,y₂,...,yₜ. |
| • | Design length = n. |

Output:

| | |
|---|---|
| • | \hat{x} - Estimation of the original sequence. |

| | |
|---|---|
| 1. | G = ∅ |
| 2. | Use Algorithm 8 and Algorithm 9, with C,n,k = 2 as parameters, to compute |
| | a multiset of candidates. Save the candidates and their frequencies in it in S. |
| 3. | If S has one or more sequence of length n, return one that minimizes the sum |
| | of edit distance to the traces in C. |
| 4. | If S has one or more sequences of length n − 1 or n + 1, return the sequence is |
| | most frequent in the multiset S (if there is more than one choose randomly). |
| 5. | Return the first sequence in S. |

i₁^{x} [symbol] i₂^{x} [symbol] . . . [symbol] .

Furthermore, we also define the embedding sequence of w in x, denoted by u_{x,w}, as a sequence of length |x| where for 1 [symbol] j [symbol] [symbol] , u_{x,w}(iⱼ^{x}) equals to x(iⱼ^{x}) and otherwise it equals to ?. ³ A subsequence can have more than one set of such indices, while the number of such sets is defined as the embedding number (see more details in the work by Elzinga et al.). In our algorithm, we chose one of these sets arbitrarily.

The gap of x, y and their LCS sequence w in index 1 [symbol] j [symbol] |x| with respect to u_{z,w} and u_{y,w}, denoted by gapᵤ_{_{x,w,}}ᵤ_{_{x,w}}(j), is defined as follows. In case the j-th or the (j−1)-th symbol in u_{x,w} equals ?, gapᵤ_{_{x,w,}}ᵤ_{_{y,w}}(j) is defined as an empty sequence. Otherwise, the symbol u_{x,w}(j) also appears in w. Denote by j′, the index of the symbol u_{x,w}(j) in w. The sequence w is an LCS of x and y, and u_{y,w} is the embedding sequence of w in y. Given u_{y,w}, we can define one set of indices such that i₁^{v} [symbol] i₂^{y} [symbol] . . . [symbol] [symbol] , such that w(j′)=y(i_{j′}^{v}) for 1 [symbol] j′ [symbol] [symbol] . Given such a set of indices, gapᵤ_{_{x,w,}}ᵤ_{_{y,w}}(j) is defined as the sequence y_{[i}_{_{j′−1}}_{^{v}}_{+1:i}_{_{j′}}_{^{y}}_{-1]}, which is the sequence between the appearances of the j′-th and the (j′−1)-th symbols of w in y. Note that since i_{j′}^{v} can be equal to i_{j′-1}^{v}+1, gapᵤ_{_{x,w,}}ᵤ_{_{y,w}}(j) can be an empty sequence.

The pattern of x and y with respect to the LCS sequence w, its embedding sequences u_{x,w} and u_{y,w}, an index 1 [symbol] i [symbol] |x| and a length m [symbol] 2, denoted by Ptn(x,y,w,u_{x,w},u_{y,w},i,m), is defined as: Ptn(x,y,w,u_{x,w},u_{y,w},i,m) [symbol] (u_{x,w}(i−1),gapᵤ_{_{x,w,}}ᵤ_{_{y,w,}}ᵥ(i),u_{x,w}(i), . . . , gapᵤ_{_{x,w,}}ᵤ_{_{y,w,}}_{y}(i+m−1). where for i<1 and i>|x|, the symbol u_{x,w}(i) is defined as the null character and gapᵤ_{_{x,w,}}ᵤ_{_{y,w,}}_{y}is defined as an empty sequence.

We also define the prefix and suffix of a pattern Ptn(x,y,w,u_{x,w},u_{y,w},i,m) to be:
Prefix(Ptn(x,y,w,u_{x,w},u_{y,w},i,m)) [symbol] (u_{x,w}(i−1), gapᵤ_{_{x,w,}}ᵤ_{_{y,w,}}_{y}(i), u_{x,w}(i), . . . , u_{x,w}
Suffix(Ptn(x,y,w,u_{x,w},u_{y,w},i,p) [symbol] (u_{x,w}(i), gapᵤ_{_{x,w,}}ᵤ_{_{y,w,}}_{y}(i+1), . . . , u_{x,w}(i+m−1)).

Finally, we define

P(x,y,w,u_{x,w},u_{y,w},m) [symbol] {Ptn(x,y,w,u_{x,w},u_{y,w}, , i,m):1 [symbol] i [symbol] |x|}.

Algorithm 7 receives a cluster C of t traces and one of the traces in the cluster yₖ. First, the algorithm initializes L[yₖ], which is a set of |yₖ| empty lists. For 1 [symbol] i [symbol] |yₖ|, the i-th list of L[yₖ] is denoted by L[yₖ]ᵢ. Algorithm 7 pairs yₖ with each of the other traces in C. For each pair of traces, yₖ and yₕ, Algorithm 7 computes an arbitrary LCS sequence w, and an arbitrary embedding sequence u_{y}_{_{k,}}_{w}. Then it uses w and u_{y}_{ₖ}_{,w} to computes P(yₖ, yₕ, w, u_{y}_{ₖ}_{,w}, u_{y}_{_{h,}}_{w}, m). For 1 [symbol] i [symbol] |yₖ|, the algorithm saves Ptn(yₖ, yₕ, w, u_{y}_{_{k,}}_{w}, u_{y}_{_{h,}}_{w},i, m) in L[yₖ]ᵢ. Then, Algorithm 7 builds the pattern graph Gₚₐₜ(yₖ)=(V(yₖ), E(yₖ)), which is a directed acyclic graph, and is defined as follows.
1. V(yₖ)={((Ptn(yₖ, yₕ, w, u_{y}_{_{k,}}_{w}, i, m), i):1 [symbol] h [symbol] t, h≠k, 1 [symbol] i [symbol] |yₖ|}∪{S, U}.
The vertices are pairs of pattern and their index. Note that the same pattern can appear in several vertices with different indices i. The value |V| equals to the number of distinct pattern-index pairs.
2. E(yₖ)={e=(v, u):v=(Ptn(yₖ, yₕ, w, u_{y}_{_{k,}}_{w}, i, m), i), u=(Ptn(yₖ, yₕ, w, u_{y}_{_{k,}}_{w}, u_{y}_{_{h,}}_{w}, i+1, m), i+1),
Suffix(Ptn(yₖ, yₕ, w, u_{y}_{_{k,}}_{w}, u_{y}_{_{h,}}_{w}, i, m)=Preffix(Ptn(yₖ, yₕ, w, u_{y}_{_{k,}}_{w}, u_{y}_{_{h,}}_{w}, i+1, m)}.
3. The weights of the edges are defined by w: E→N as follows:
For e=(v, u), where u=(Ptn(yₖ, yₕ, w, u_{y}_{_{k,}}_{w}, u_{y}_{_{h,}}_{w}, i, m), i), it holds that

w(e)=|{Ptn∈L[yₖ]ᵢ:Ptn=Ptn(yₖ,yₕ,w,u_{y}_{ₕ}_{,w},i,m}|,

which is the number of appearances of Ptn(yₖ, yₕ, w, u_{y}_{_{k,}}_{w}i, m) in L[yₖ]ᵢ.
4. The vertex S which does not correspond to any pattern, is connected to all vertices of the first index. The weight of these edges is the number of appearances of the incoming vertex pattern.

5. The vertex U has incoming edges from all vertices of the last index and the weight of each edge is zero.

Algorithm 7 finds a longest path from S to U in the graph. This path induces a sequence, denoted by ŷₖ, that consists of patterns of yₖ where some of them include gaps. The algorithm returns ŷₖ, which is a revised version of yₖ, while adding to the original sequence of yₖ the gaps that are inherited from the longest path vertices.

Example 1. We present here a short example of the definitions above and Algorithm 7. The original strand in this example is x and the cluster of traces is C=y₁, . . . , y₅, Note that the original length is n=10. The traces are noisy copies of x and include deletions, insertions, and substitutions. In this example Algorithm 7 receives the cluster C and the trace yₖ=y₁ as its input.

x = GTAGTGCCTG.
y₁ = GTAGGTGCCG.
y₂ = GTAGTCCTG.
y₃ = GTAGTGCCTG.
y₄ = GTAGCGCCAG.
y₅ = GCATGCTCTG.

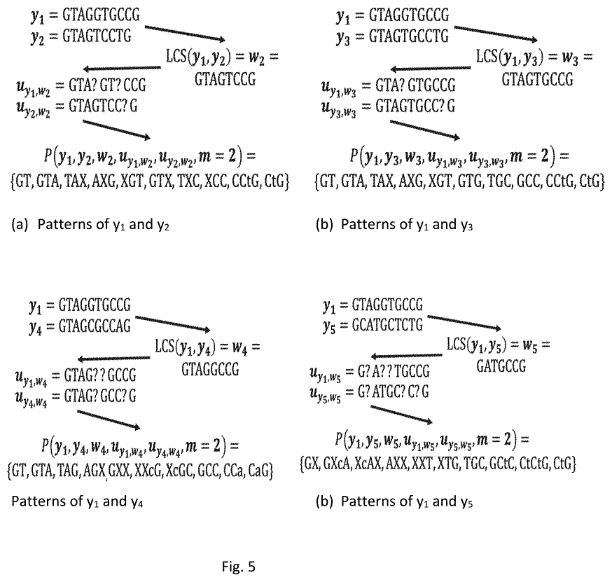

FIG. 5 presents the process of computing the patterns of (y₁, y₂), (y₁, y₃), (y₁, y₄), (y₁, y₅). For each pair, y₁ and yᵢ, FIG. 5 depicts wᵢ, which is an LCS of the sequences y₁ and yᵢ. Then, the figure presents u_{y}_{_{1,}}_{w}_{ᵢ} and u_{y}_{ᵢ}_{i,w}_{ᵢ}, which are the embedding sequences that Algorithm 7 uses in order to compute the patterns. Lastly, the list of patterns of each pair is depicted in an increasing order of their indices. Note that lowercase symbols present gaps and X presents the symbol ?. The following list summarizes the patterns and their frequencies. Each list includes patterns from specific index. The numbers on the right side of each pattern in a list represents the pattern's frequency.

L[y₁]₁={GT:3, GX:1}.

L[y₁]₂={GTA:3, GXcA:1}.

L[y₁]₃={TAX:2, TAG:1, XcAX:1}.

L[y₁]₄={AXG:2, AGX:1, AXX:1}.

L[y₁]₅={XGT:2, GXX:1, XXT:1}.

L[y₁]₆={GTG:1, GTX:1, XXcG:1, XTG :1}.

L[y₁]₇={TGC:2, TXC:1, XcGC:1}.

L[y₁]₈={GCC:2, SCC:1, GCtG:1}.

L[y₁]₉={CCtG:2, CCa:1.CtCtG:1}.

L[y₁]₁₀={CtG:3, CaG:1}.

It is not hard to observe that the longest path in the pattern path graph of this example is:

GT→GTA→TAX→AXG→XGT→GTG→TGC→GCC→CCtG→CtG→G,

and the algorithm output will be ŷ₁ =GTAGTGCCTG=x.

In this section we present an evaluation of the accuracy of Algorithm 4 and Algorithm 5 on simulated data and on data from previous DNA storage experiments. We also implemented the lookahead algorithms by gopalan et al.⁴ and by viswanathan et al.⁵, and our variation of the BMA algorithm to support also insertion and substitution errors, which is referred by the Divider BMA algorithm. ⁴The parameters we used for the window size w of the algorithm were 2 [symbol] w [symbol] 4, and we present the results for all of them.⁵The parameters we used for the algorithm by viswanatan et al. were [symbol] =5, δ=(1+pₛ)/2, r=2 and γ=¾, while for the data of the DNA storage experiments the substitution probability pₛ was taken from SOLQC

Algorithm 6 Error Vectors Majority

Input:

| | |
|---|---|
| • | Cluster C of t noisy traces: y₁,y₂,...,yₜ. |
| • | Design length = n |
| • | yᵢ ∈ C - a copy from the cluster. |

Output:

| | |
|---|---|
| • | \hat{x} - a revised version of yᵢ, an estimation of yᵢ with less |
| | substitution and insertion errors. |

| 1. | S = “”, an empty vector. | |
|---|---|---|
| 2. | for yₖ ∈ C, k ≠ i do | |
| | (a) | Compute EV(yᵢ,yₖ). |
| | end for | |
| 3. | for 1 [symbol] j [symbol] \|yᵢ\| + 1 do | |
| | (a) | Set S(j) to be the operation that appeared in the j-th entry |
| | | of most of the EV that computed in Step 2. |

| | |
|---|---|
| | end for |
| 4. | Perform the operations from the vector S on yᵢ and save the |
| | resulted sequence in \hat{x}. |
| 5. | Return \hat{x}. |

Algorithm 7 Pattern-Path

Input:

| | |
|---|---|
| • | Cluster C of t noisy traces: y₁,y₂,...,yₜ. |
| • | Design length = n |
| • | yₖ ∈ C - a copy from cluster C. |

Output:

| | • | ŷₖ - a revised version of yₖ. The sequence ŷₖ consists of yₖ's original sym- | |
|---|---|---|---|
| | | bols and also includes some additional symbols, which are estimations of the | |
| | | symbols deleted from yₖ. | |
| 1. | L[yₖ] = {L[yₖ]₁,..., L[yₖ]_{\|y}^{ₖ}_{\|}}, a list of \|yₖ\| empty lists, where each represents | | |
| | the list of patterns before the symbol i in yᵢ, where the last list represents | | |
| | symbols before the end of the sequence. | | |
| 2. | /*In this stage we pair yₖ with all the copies from the cluster, cre- | | |
| | ate list L[yₖ] of \|yₖ\| lists of patterns of symbol i and their frequen- | | |
| | cies*/ | | |
| | for yₕ ∈ C do | | |
| | | (a) | Compute w an LCS sequence of yₖ,yₕ. |
| | | (b) | Compute u_{y}^{ₖ}_{,w} an embedding sequence for yₖ and w . |
| | | (c) | Computes P(yₖ,yₕ,w,u_{y}^{ₖ}_{,w},m = 3). |
| | | (d) | For each 1 [symbol] i [symbol] yₖ add to L[yₕ]ᵢ the pattern |
| | | | P(yₖ,yₕ,w,u_{y}^{ₖ}_{,w},i,3). |

| | |
|---|---|
| | end for |
| 3. | Build Gₚₐₜ = (V,E) - the pattern graph. |
| 4. | Find the longest path from the source vertex S in Gₚₐₜ. |
| 5. | Let ŷₖ bet the sequence that inherited from the patterns of the |
| | vertices of the longest path. |
| 6. | Return ŷₖ. |

Algorithm 8 Iterative Reconstruction-Horizontal

Input:

| | |
|---|---|
| • | Cluster C of t noisy traces: y₁,y₂,...,yₜ. |
| • | Design length = n. |

Output:

| | • | S={s₁,s₂,...,sₚ}, a multiset of p candidates, that estimate the original se- | | |
|---|---|---|---|---|
| | | quence of the cluster. | | |
| 1. | S = ∅, C_{orig} = C | | | |
| 2. | for j= 1, 2, ..., k do | | | |
| | | (a) | Cₜₘₚ = ∅ | |
| | | (b) | for yᵢ ∈ C_{orig} do | |
| | | | i. | Perform Algorithm 6 on yᵢ to correct substitution errors. |
| | | | ii. | Perform Algorithm 7 on yᵢ to correct deletion errors. |
| | | | iii. | Perform Algorithm 6 on yᵢ to correct insertion errors. |
| | | | iv. | Cₜₘₚ = Cₜₘₚ∪{yᵢ}. |

| | |
|---|---|
| (c) | end for |
| (d) | C = Cₜₘₚ. |

| | |
|---|---|
| | end for |
| 3. | S = S ∪ C. |
| 4. | Set C_{orig} = C_{orig}^{R} and repeat Steps 2-3 on C_{orig}^{R}. Add the results to S. |

Algorithm 9 Iterative Reconstruction-Vertical

Input:

| | |
|---|---|
| • | Cluster C of t noisy traces: y₁,y₂...,yₜ. |
| • | Design length = n. |

Output:

| | • | S={s₁,s₂,...,sₚ}, a multiset of p candidates, sequences that estimates the | | |
|---|---|---|---|---|
| | | original sequence of the cluster. | | |
| 1. | S = ∅, C_{orig} = C | | | |
| 2. | for j= 1, 2, ..., k do | | | |
| | | (a) | Cₜₘₚ = ∅ | |
| | | (b) | for yᵢ ∈ C do | |
| | | | i. | Perform Algorithm 6 on yᵢ to correct substitution errors. |
| | | | ii. | Cₜₘₚ = Cₜₘₚ ∪ {yᵢ}. |
| | | (c) | end for | |
| | | (d) | C = Cₜₘₚ | |
| | | (e) | Cₜₘₚ = ∅ | |
| | | (f) | for yᵢ ∈ C do | |
| | | | i. | Perform Algorithm 7 on yᵢ to correct deletion errors. |
| | | | ii. | Cₜₘₚ = Cₜₘₚ ∪ {yᵢ}. |
| | | (g) | end for | |
| | | (h) | C = Cₜₘₚ | |
| | | (i) | Cₜₘₚ = ∅ | |
| | | (j) | for yᵢ ∈ C do | |
| | | | i. | Perform Algorithm 6 on yᵢ to correct insertion errors. |
| | | | ii. | Cₜₘₚ = Cₜₘₚ ∪ {yᵢ}. |

| | |
|---|---|
| (k) | end for |
| (l) | C = Cₜₘₚ |

end for
3. S = S∪C
4. Set C_{orig} = C_{orig}^{R} and repeat Steps 2-3 on C_{orig}^{R}. Add the results to S.

The Divider BMA algorithm receives a cluster and the design length n. The Divider BMA algorithm divides the traces of the cluster into three sub-clusters by their lengths, traces of length n, traces of length smaller than n and traces of length larger than n. It performs a majority vote on the traces of length n. Then, similarly to the technique presented in the BMA algorithm and in the lookahead algorithm by Gopalan et al., the Divider BMA algorithm performs a majority vote on the subcluster of traces of length smaller than n, while detecting and correcting deletion errors. Lastly, the Divider BMA algorithm uses the same technique on the traces of length larger than n to detect and correct insertion errors.

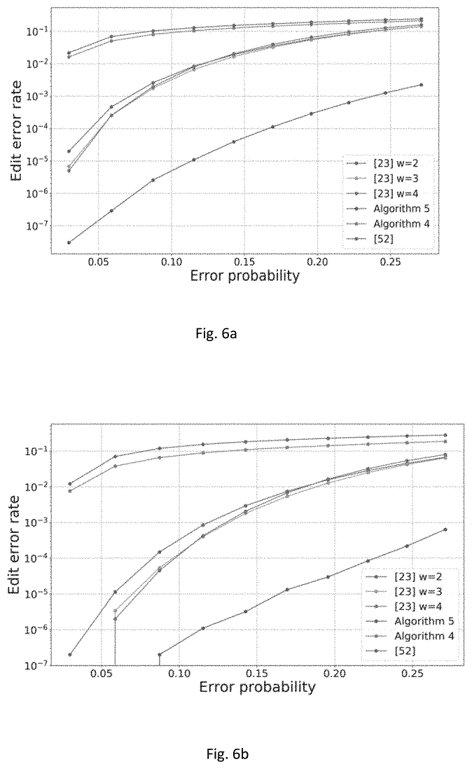

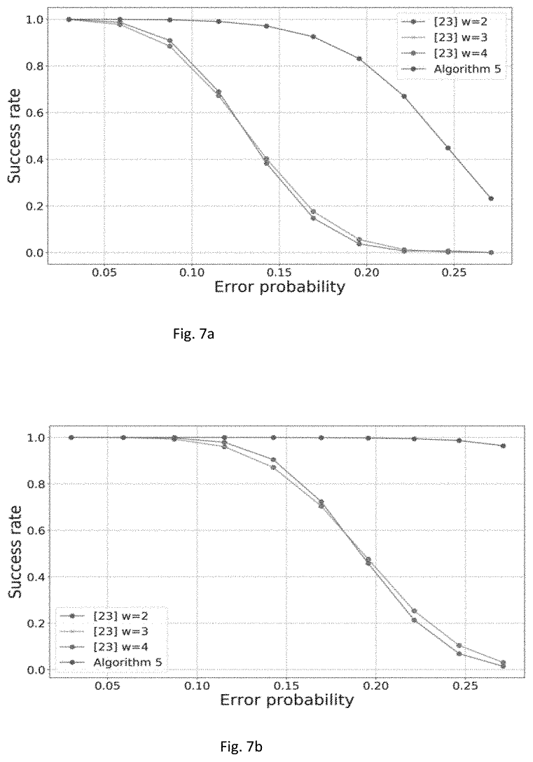

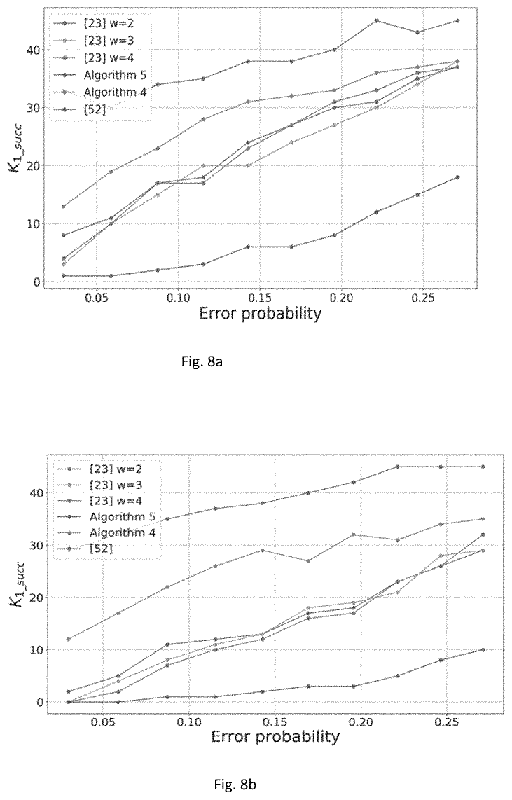

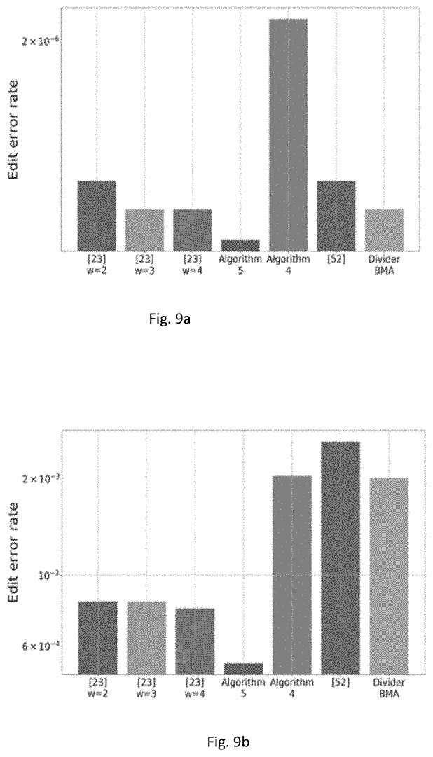

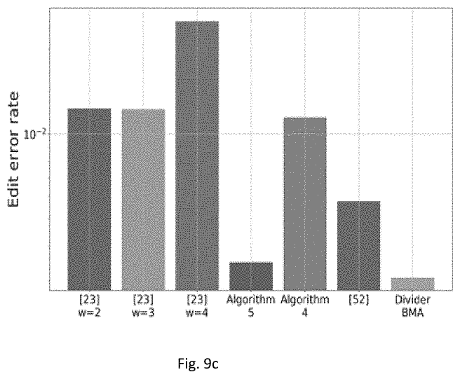

We compare the edit error rates and the success rates of all the algorithms. In all of the simulations, Algorithm 5 presented significantly smaller edit error rates and higher success rates. The results on the simulated data are depicted in Figure 6, FIG. 7, and FIG. 8. The results on the data from previous DNA storage experiments can be found in FIG. 9.

### 0.14 Results on Simulated Data

We evaluated the accuracy of Algorithm 4 and Algorithm 5 by simulations. First, we present our interpretation of the deletion-insertion-substitution channel. In our deletion-insertion-substitution channel, the sequence is transmitted symbol-by-symbol. First, before transmitting the symbol, it checks for an insertion error before the transmitted symbol. The channel flips a coin, and in probability pᵢ, an insertion error occurs before the transmitted symbol. If an insertion error occurs, the inserted symbol is chosen uniformly. Then, the channel checks for a deletion error, and again flips a coin, and in probability p_{d} the transmitted symbol is deleted. Lastly, the channel checks for a substitution error. The channel flips a coin, and in probability pₛ the transmitted symbol is substituted to another symbol. The substituted symbol is chosen uniformly. In case that both deletion and substitution errors occurs in the same symbol, we refer to it as a substitution.

We simulated 100,000 clusters of sizes t=6, 10, 20, the sequences length was n=100, and the alphabet size was q=4. The deletion, insertion, and substitution probabilities were all identical, and ranged between 0.01 and 0.1. It means that the actual error probability of each cluster was 1−(1−pᵢ)(1−pₛ)(1−p_{d}) and ranged between 0.029701 and 0.271. We reconstructed the original sequences of the clusters using Algorithm 4, Algorithm 5 and the algorithms from gopalan and from viswanathan. For each algorithm we evaluated its edit error rate, the success rate, and the value of k_{1_succ}. The edit error rate of Algorithm 5 was the lowest among the tested algorithms, while the algorithm from Viswanathan presented the highest edit error rates. Moreover, it can be seen that Algorithm 5 presented significantly low edit error rates value for higher values of error probabilities. In addition, Algorithm 5 also presented the lowest value of k_{1_succ}. For example, when the cluster size was t=20 and the error probability was p=0.142625, the value of k_{1_succ} of Algorithm 5 was 2, while the other algorithms presented k_{1-succ} values of at least 12. The results of these simulations for cluster sizes of t=10 and t=20 can be found in FIG. 6, FIG. 7 and FIG. 8.

### 0.15 Results on Data from DNA Storage Experiments

In this section we present the results of the tested algorithms on data from previous DNA storage experiments. The clustering of these data sets was made by the SOLQC tool. We performed each of the tested algorithms on the data and evaluated the edit error rates. Note that in order to reduce the runtime of Algorithm 5 we filtered clusters of size t>25 to have only the first 25 traces. Also here, Algorithm 5 presented the lowest edit error rates in almost all of the tested data sets. These results are depicted in FIG. 9.

### 0.16 Performance Evaluation

We evaluated the performance of the different algorithms discussed in this paper. The performance evaluation was performed on our server with Intel(R) Xeon(R) CPU E5-2630 v3 2.40 GHz. We implemented our algorithms as well as the previously published algorithm from Gopalan, which presented the second-lowest error rates in our results from Section 0.14. In order to present reliable performance evaluation, the clusters in our experiments were reconstructed in serial order. However, it is important to note, that for practical uses, additional performance improvements can be made by performing the algorithms on the different clusters in parallel and shortening the running time.
Experiment A included performing reconstruction of simulated data of 2,000 clusters of sequence length of n=100, p_{d}=pₛ=pᵢ=0.05, so the total error rate was 0.142625. The cluster sizes were distributed uniformly between t=1 and t=40. The Gopalan algorithm (with parameter w=3) reconstructed the full data set in one second with total error rate of 0.03028. Algorithm 5 reconstructed the 2,000 clusters in 2,887 seconds and presented roughly 50% less errors with total edit error rate of 0.014925.
Experiment B included performing full reconstruction of the data set from GHP15. Algorithm 5 reconstructed the full data set in 9, 768 seconds with total edit error rate of 0.00053, while the algorithm from Gopalan (with parameter w=3) reconstructed the full data set in 50 seconds with total error rate of 0.00081, so it presented approximately 53% more errors compare to Algorithm 5.
Experiment C included performing reconstruction on 200,000 clusters from the data set of OAC17. Algorithm 5 reconstructed the clusters in 456, 200 seconds and presented total edit error rate of 0.00352, while the algorithm from Gopalan (with parameter w=3) reconstructed the clusters in 296 seconds and total edit error rate of 0.012, which is approximately 3.4 times more errors. Since for this data set, the divider BMA presented the lowest error rate, we decided to evaluate its performance on this data set. The divider BMA algorithm reconstructed the data set in 234 seconds and error rate of 0.0031.
   Lastly, to improve the performance of Algorithm 5, we created a hybrid algorithm, that invokes the algorithm from Gopalan on clusters with more than 20 traces, and otherwise it invokes Algorithm 5. The results of the hybrid algorithm on the simulated data from the experiment A had an edit error rate of 0.02 and in 330 seconds for the first experiment of the simulated data. Since in data from previous DNA storage experiments the variance in the cluster size and in the error rates can be really high, we added an additional condition to the hybrid algorithm, so it invokes the algorithm from Gopalan if the cluster is of size 20 or larger, or if the absolute distance of the difference between the average length of the traces in the cluster and the design length is larger than 10% of the design length. The hybrid algorithm reconstructed the full data set of GHP15 (experiment B) in 37 seconds and presented error rate of 0.000676. We also performed the hybrid algorithm on 200,000 clusters from the data set of (experiment A). The hybrid algorithm reconstructed these 200,000 clusters in 82, 508 seconds, with edit error rate of 0.002295. The results of the performance experiments are also depicted in Table 1.

TABLE 1
Performance evaluation of Algorithm 5, Gopalan et al. algorithmGopalan,
and the hybrid algorithm. The number presented the running
time in seconds and the error rate of each of the algorithms
for each of the experiments.

| | Algorithm | Gopalan et | Hybrid |
|---|---|---|---|
| | 5 | al. Gopalan | algorithm |
| Experiment A - | 2,887 | 1 | 330 |
| time (sec.) | | | |
| Experiment A - | 0.014925 | 0.03028 | 0.02 |
| error rate | | | |
| Experiment B GHP15- | 9,768 | 50 | 37 |
| time (sec.) | | | |
| Experiment B GHP15- | 0.00053 | 0.00081 | 0.000676 |
| error rate | | | |
| Experiment C OAC17- | 456,200 | 296 | 82,508 |
| time (sec.) | | | |
| Experiment C OAC17- | 0.00352 | 0.012 | 0.002295 |
| error rate | | | |

We presented in this paper several new algorithms for the deletion DNA reconstruction problem and for the DNA reconstruction problem. While most of the previously published algorithms use a symbol-wise majority approaches, our algorithms look globally on the entire sequence of the traces, and use the LCS or SCS of a given set of traces. Our algorithms are designed to specifically support DNA storage systems and to reduce the edit error rate of the reconstructed sequences. According to our tests on simulated data and on data from DNA storage experiments, we found out that our algorithms significantly reduced the error rates compared to the previously published algorithms. Moreover, our algorithms performed even better when the error probabilities were high, while using less traces than the other algorithms. Even though our algorithms improved previous results, there are still several challenges that need to be addressed in order to fully solve the DNA reconstruction problem. Some of these challenges are listed as follows.

### 0.17 The CPL Algorithm

In this section we present Algorithm 0.17. Algorithm 0.17 receives cluster of t traces C, the design length n, and a trace from the cluster, yⱼ∈C. The algorithm calculates for each 1 [symbol] k [symbol] t, k≠i, the vector EV(yⱼ, yₖ), which is a vector of edit operations to transform yⱼ to yₖ. The set of such vectors is denoted as EVᵥ_{ⱼ} (C). Each entry of the vector EV(yⱼ, yₖ) consists of an operation and a symbol, where the operation is either copy, substitute, delete, or insert, and the symbols represents the symbol(s) of the operation. Symbols of insertions operation are considered out-of-order operation and are marked accordingly. As mentioned above, in some of the cases the vector EV (yⱼ, yₖ) is not unique, in this case, Algorithm 0.17, chooses one of the vectors randomly. We further denote EVᵤ_{ⱼ}(C)[i] as the set of the symbols in the i-th index of each error vector in EVᵥ_{ⱼ}(C). As mentioned above, since inserted symbols are omitted, the set EVᵥ_{ⱼ}(C)[i] is refer to symbols in the i-th index of the vectors in EVᵥ_{ⱼ}(C), while the index i is calculated only by counting the non-inserted symbols. Then, the algorithm uses the set of error vector to compute the conditional probability of each symbol per index. For each index 1 [symbol] i [symbol] n and for any two symbols u, v∈{A, C, G, T}, we define the conditional probability of symbol v in index i+1, given that the i-th symbol is u.

$${P}_{i}^{{y}_{j} , 𝒞} ( v \vert u ) := \frac{\vert v ∈ E {V}_{{y}_{j}} ( 𝒞 ) , v [ i ] = v and v [ i + 1 ] = u \vert}{\vert \{ v ∈ E {V}_{{y}_{j}} ( 𝒞 ) , v [ i ] = v \} \vert} ,$$

where v[i] referes to the i-th symb of the vector v. Following that, the algorithm calculates the set Post_{i,u}^{v}^{,C}=v|v∈EV_{y}_{ⱼ}(C)[i], Pᵢ^{y}^{ⱼ}^{,C}(v|u)≠0, which is the set of the symbols that can be achieved on the i-th index of an error vector in EVᵥ_{ⱼ}(C), given that the i-th symbols was u. Note that, the set Post_{i,u}^{v}^{ⱼ}^{,C} can be empty.

Based on these probabilities, we can now define the edit graph Gᵥ_{ⱼ}_{,c}=(V(yⱼ), E(yⱼ)) of a trace yⱼ, the edit graph of the sequence is defined as follows:
1. V(yⱼ)={u|u∈EV_{y}_{ⱼ}(C)[i], 1 [symbol] <i [symbol] 2|yⱼ|+1}∪{0}—the vertices are defined as the symbols from the sets EVᵥ_{ⱼ}(C).
2. E(y₃)={(u, v)ᵢ|u∈EVᵥ_{ⱼ}(C)[i], v∈Post_{i,u}^{y}^{ⱼ}^{,C}}∪{(u, 0)ᵢ∈EVᵥ_{ⱼ}(C)[i], Post_{i,u}^{v}^{ⱼ}^{,C}=0}—each edge connects between any two vertices that represents symbols that appears consecutively in an error vector in the set EV_{y}_{ⱼ}(C).
3. W:E(yⱼ)→ [symbol] —weight function, defined on the edges. The weight of edge (u, v)ᵢ∈E(yⱼ) is defined as the log of its conditional probability, that is W((u, v)ᵢ)=log(Pᵢ^{y}^{ⱼ}^{,C}(v|u)).

Finally, Algorithm 0.17 calculates the heaviest path of length n+1 in the edit graph G_{y}_{ⱼ}_{,c}=(V(yⱼ), E(yⱼ)). There are n+1 vertices in this path, which contains n symbols and 1 vertex of the symbol 0. Therefore, the path induce a sequence \hat{x} that estimates the original sequence of the cluster. The output of Algorithm 0.17 is \hat{x}.

Algorithm 10 Conditional Probability Algorithm

```
Input:
    • Cluster C of t noisy traces: y₁,y₂,...,yₜ.
    • Design length = n
    • yⱼ ∈ C - a copy from cluster C.
Output:
    • \hat{x} - an estimation of the original sequence x.
  1. Compute the set EV_{y}^{ⱼ}( [symbol]   ), where the length of vectors is bounded by 2|yⱼ| + 1.
  2. For 1 [symbol]   i [symbol]   2|yⱼ| + 1, and for any two symbols u,v calculate [symbol]  (v|u).
  3. Build the edit graph [symbol]  .
  4. Compute p the longest path of length n + 1 in [symbol]  .
  5. Return the labels of the vertices of the path \hat{x} = p.
```

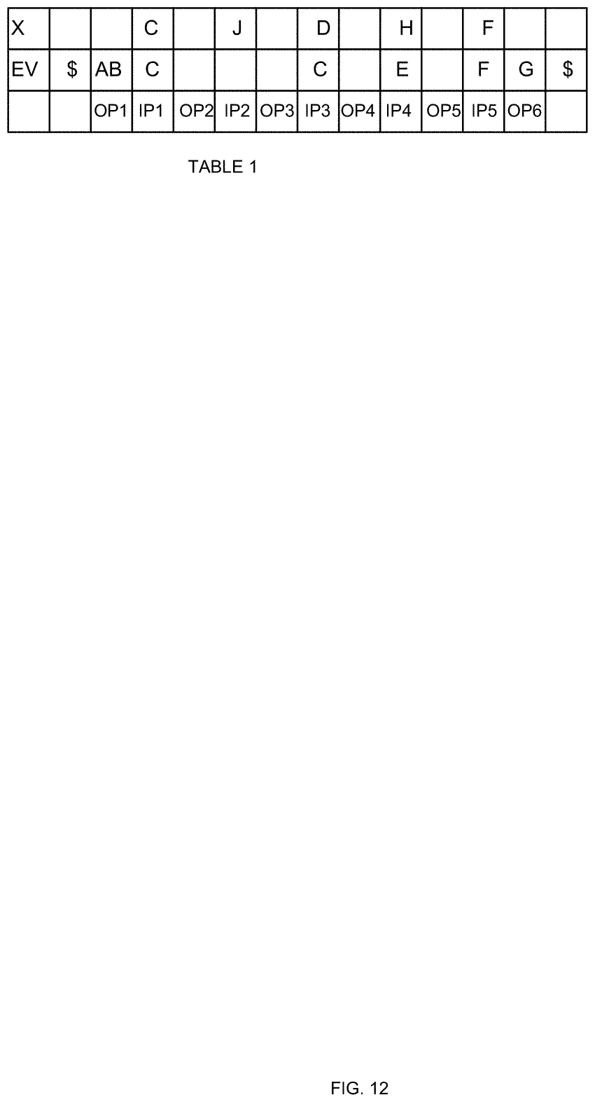

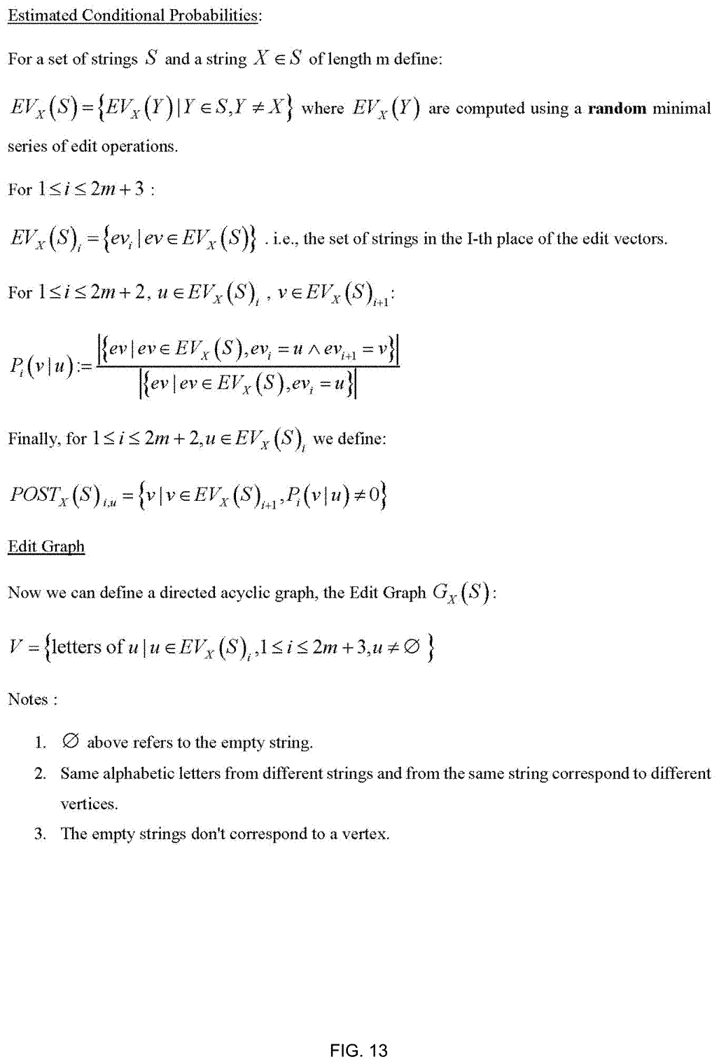

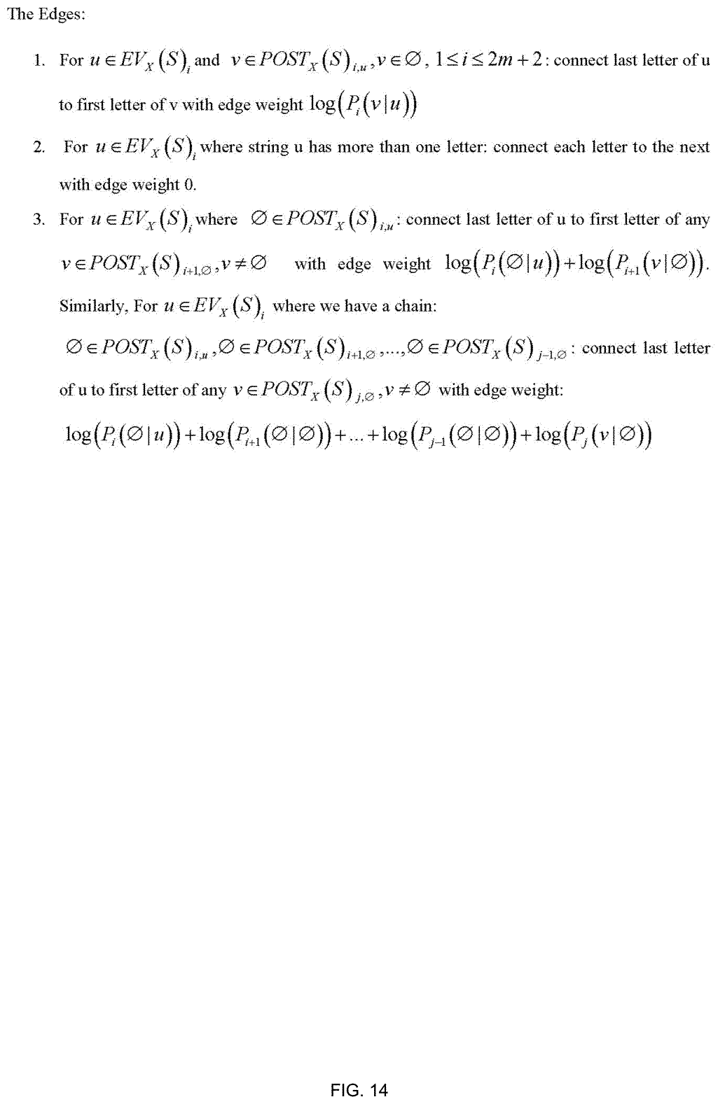

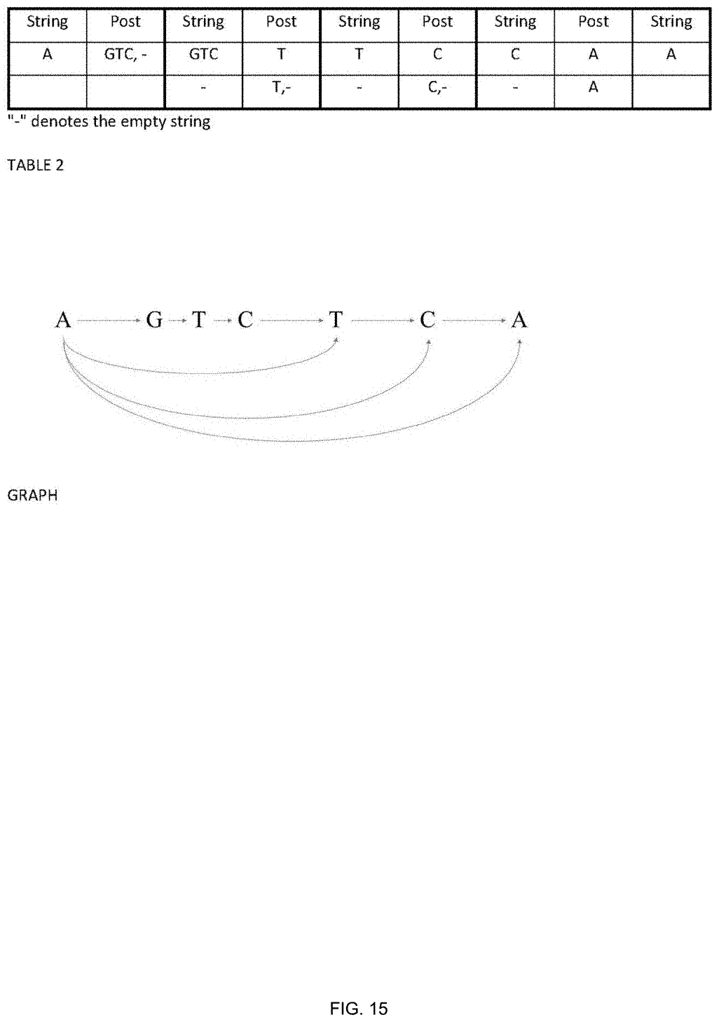

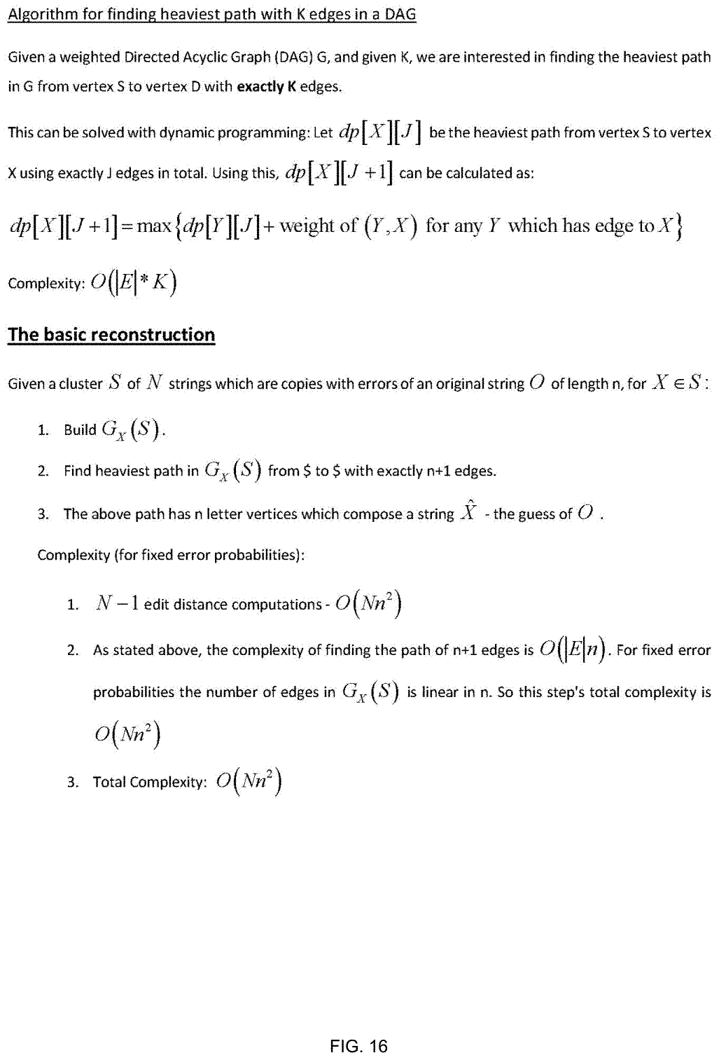

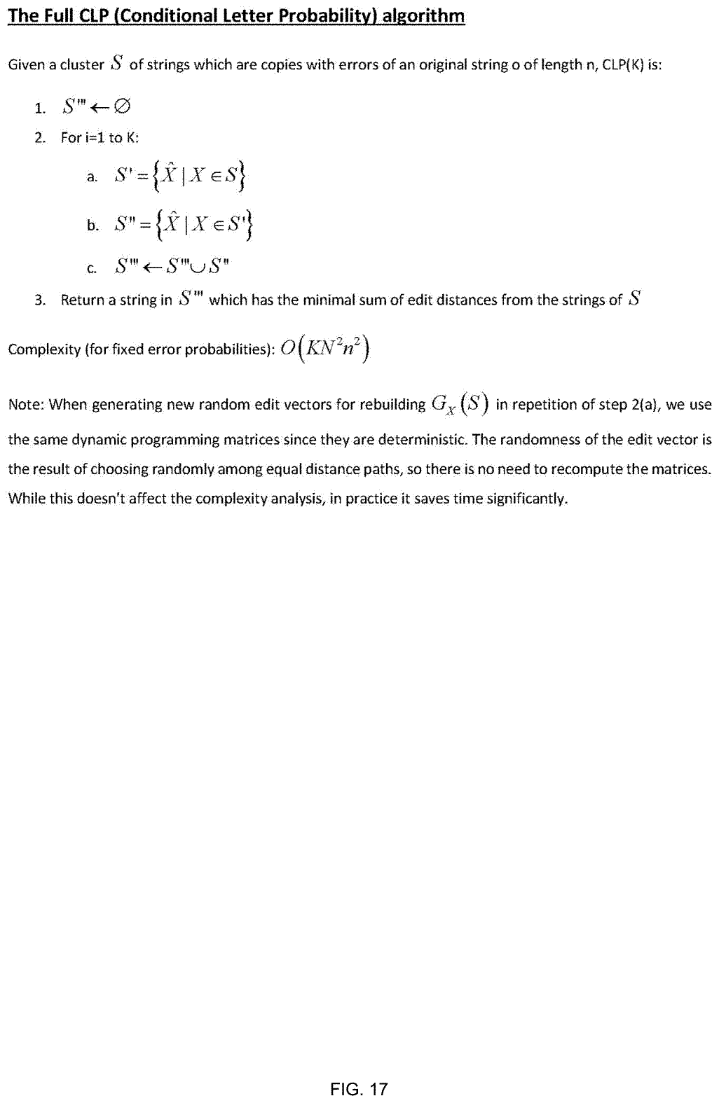

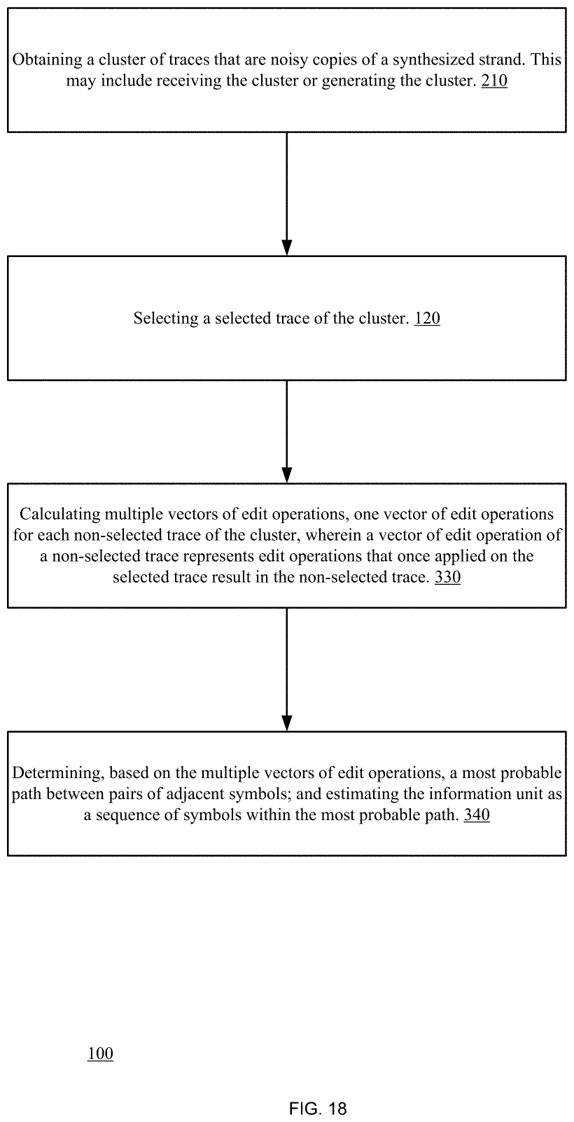

Edit Vector When editing string X to string Y, we use 4 operations: insert letters of Y before a letter of X, delete a letter of X, substitute a letter of X for a letter of Y, copy a letter of X. These operations can be divided into two categories: In-place edit operations: operations on a letter of x: delete the letter, substitute it or copy it. Out-of-place edit operations: insert a subtracting of Y before a letter of X. If X is a string of length m, we can regard any series of edit operations which edit X to string Y as edit strings: m strings for in-place operations on each letter of X and m+1 strings for out-of-place operations, i.e. inserts, before each letter of X and after the last one (before end of string). In-place edit strings: copy letter x of X: x substitute letter x of X for letter y of Y: y delete letter x of X: empty string Out-of-place edit strings: insert string s before letter of X: s (empty string for no insert) So, given a series of edit operations which edit X to string Y ,for we define a vector v of strings of length : in place the edit string of the in-place operation of letter of X. in place the edit string of the out-of-place operation of letter of X. In place the edit string of the before the end insert. Finally, we can define the edit vector of X to Y. Example: X=CJDHF, Y=ABCDEFG We can edit X to Y with the following operations: Insert AB before C, Copy C, Delete J, Copy D, Substitute H with E, Copy F, Insert G before end. The resulting Graph is illustrated in FIG. 12. An example of estimated conditional probabilities is illustrated in FIGS. 13 and 14. An example of a second table and a graph related to a symbol located at a certain location are illustrated in FIG. 15. FIG. 16 is an example of a method for finding the heaviest path. FIG. 17 is an example of a method for finding a conditional letter probability. And FIG. 18 illustrates an example of a method 300 for estimating an information unit. Method 300 is for estimating an information unit represented by a cluster of traces that are noisy copies of a synthesized strand. Method 300 may start by step 310 of obtaining a cluster of traces that are noisy copies of a synthesized strand. This may include receiving the cluster or generating the cluster. Step 310 may be followed by step 320 of selecting a selected trace of the cluster. Any selection method or parameter may be used. Step 320 may be followed by step 330 of calculating multiple vectors of edit operations, one vector of edit operations for each non-selected trace of the cluster, wherein a vector of edit operation of a non-selected trace represents edit operations that once applied on the selected trace result in the non-selected trace. Step 330 may be followed by step 340 of determining, based on the multiple vectors of edit operations, a most probable path between pairs of adjacent symbols; and estimating the information unit as a sequence of symbols within the most probable path. The determining of the most probable path may include generating, based on the multiple vectors of edit operations, a representation of (a) possible symbols per location out of multiple locations, and (b) links between symbols of adjacent pairs of locations of the multiple locations. A location may be an index in any of the vectors, an order of the symbol within a trace, and the like. The method may include calculating an occurrence attribute of each link. The determining of the most probable path may be based on the occurrence attributes of multiple links. The occurrence attributes may be non-normalized or may be normalized - for example be normalized by a total number of links associated with a single origin symbol. The edit operations may include copy, substitution, deletion and insert. The vectors of edit operation may include symbols of edit operations, and locations of symbols on which the edit operations were applied. The method may include selecting a single vector of edit operations for a non-selected trace of the cluster when there are multiple vectors of edit operations for the non-selected trace.

In the foregoing specification, the invention has been described with reference to specific examples of embodiments of the invention. It will, however, be evident that various modifications and changes may be made therein without departing from the broader spirit and scope of the invention as set forth in the appended claims. Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality. Any arrangement of components to achieve the same functionality is effectively “associated” such that the desired functionality is achieved. Hence, any two components herein combined to achieve a particular functionality may be seen as “ associated with” each other such that the desired functionality is achieved, irrespective of architectures or intermedial components. Likewise, any two components so associated can also be viewed as being “operably connected,” or “operably coupled,” to each other to achieve the desired functionality. Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments. Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner. However, other modifications, variations and alternatives are also possible. The specifications and drawings are, accordingly, to be regarded in an illustrative rather than in a restrictive sense. In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage. While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention. It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination. It will be appreciated by persons skilled in the art that the embodiments of the disclosure are not limited by what has been particularly shown and described hereinabove. Rather the scope of the embodiments of the disclosure is defined by the appended claims and equivalents thereof.

We claim:

1. A method for estimating an information unit represented by a cluster of traces that are noisy copies of a synthesized strand, the method comprises: estimating the information unit by applying processing operations on r-tuples related to the traces, where r is smaller than a number (t) of traces of the cluster; wherein processing operations applied on at least some of the r-tuples comprise calculating a length of a shortest common supersequences (SCS) of the r-tuples.

2. The method according to claim 1 wherein the processing operations applied on at least some of the r-tuples comprise searching for a maximum likelihood SCS.

3. The method according to claim 2 wherein not finding, the maximum likelihood SCS, then returning a SCS that minimizes a sum Levenshtein distances of all the traces of the cluster.

4. The method according to claim 1 comprising repeating the processing operations for different values of r.

5. The method according to claim 4 wherein r does not exceed ten.

6. The method according to claim 4 wherein there are only a few different values of r.

7. The method according to claim 1 wherein the processing operations applied on the at least some of the r-tuples comprise calculating longest common subsequences (LCSs).

8. The method according to claim 1 wherein the estimating the information unit is based on a size of the cluster.

9. The method according to claim 1 wherein the estimating the information unit comprising estimate an error probability of the cluster using an average length of the traces.

10. The method according to claim 1 wherein the estimating the information unit comprises applying the processing operations only on a group of longest traces of the cluster.

11. The method according to claim 10 wherein the longest traces are about one fifth of the traces of the cluster.

12. The method according to claim 1 wherein a processing of a r-tuple is preceded by calculating a distance between the traces of the cluster.

13. The method according to claim 12 wherein the distance is a k-mer distance.

14. A non-transitory computer readable medium that stores instructions for:
estimating a information unit by applying processing operations on r-tuples related to traces, wherein, the information unit is represented by a cluster of the traces, the traces are noisy copies of a synthesized strand where r is smaller than a number of (t) of traces of the cluster;
wherein processing operations applied on at least some of the r-tuples comprise calculating a length of a shortest common supersequences (SCS) of the r-tuples.

15. The non-transitory computer readable medium according to claim 3 wherein the processing operations applied on at least some of the r-tuples comprise searching for a maximum likelihood SCS.

16. The non-transitory computer readable medium according to claim 15 wherein when not finding, the maximum likelihood SCS, then returning a SCS that minimizes a sum of Levenshtein distances of all the traces of the cluster.

17. The non-transitory computer readable medium according to claim 13 comprises repeating the repeating the processing operations for different values of r.

18. The non-transitory computer readable medium according to claim 17 wherein r does not exceed ten.

19. The non-transitory computer readable medium according to claim 17 wherein there are only a few different values of r.

20. The non-transitory computer readable medium according to claim 13 wherein the processing operations applied on the at least some of the r-tuples comprise calculating longest common subsequences (LCSs).

21-52. (canceled)
